(19) United States
(12) Patent Application Publication
(10) Pub. No.: US 2003/0157540 A1
(43) Pub. Date: Aug. 21, 2003

(54) Methods for isolating and characterizing short-lived proteins and arrays derived therefrom

(76) Inventors: Xianqiang Li, Palo Alto, CA (US); Xin Jiang, Saratoga, CA (US)

Correspondence Address: WILSON SONSINI GOODRICH & ROSATI, 650 PAGE MILL ROAD, PALO ALTO, CA, 943041050

(21) Appl. No.: 10/347,160

(22) Filed: Jan. 16, 2003

Related U.S. Application Data
(63) Continuation-in-part of application No. 10/053,230, filed on Jan. 16, 2002.
(63) Continuation-in-part of application No. 10/053,516, filed on Jan. 16, 2002.

(51) Int. Cl. C12Q 1/68; C12M 1/34

(52) U.S. Cl. 435/6; 435/287.2

(57) ABSTRACT

Compositions, kits and methods are provided for isolating and characterizing short-lived proteins. The method comprises: taking a library of cells, each cell in the library expressing a fusion protein comprising a reporter protein and a protein encoded by a sequence from a cDNA library derived from a sample of cells, the sequence from the cDNA library varying within the cell library; modifying a rate of protein expression or degradation by cells in the library; and selecting a population of cells from the library of cells based on the population of cells having different reporter signal intensities than other cells in the library, the difference being indicative of the population of cells expressing shorter lived fusion proteins than the fusion proteins expressed by the other cells in the library; and determining protein sequences of the fusion proteins of the selected population of cells. Also provided are oligonucleotide, protein and antibody arrays derived from short-lived proteins. The arrays can be used for efficiently profiling expression of short-lived proteins, screening for binding agents and comparing expression levels under different conditions.

### CROSS-REFERENCE TO RELATED APPLICATIONS

This application is a continuation-in-part of U.S. application Ser. No. 10/053,230, filed Jan. 16, 2002, entitled “Methods For Isolating And Characterizing Short-Lived Proteins” and U.S. application Ser. No. 10/053,516, filed Jan. 16, 2002, entitled “Screening Methods Involving The Detection of Short-Lived Proteins.” The above applications are hereby incorporated by reference.

### FIELD OF THE INVENTION

The present invention relates to detecting and characterizing proteins and more specifically to detecting and characterizing short-lived proteins, and nucleic acid or protein arrays derived from the short-lived proteins.

### DESCRIPTION OF RELATED ART

The availability of the entire human genome sequence will revolutionize the way biology and medicine will be explored in the next century and beyond. However, the next big challenge is the development of technologies for the comprehensive analysis of gene expression and the interpretation of the functionality of individual genes and their gene products in the human genome.

A gene is genetic information (i.e., DNA or RNA) that encodes a protein. Proteins, the expression product of genes, have different biological functions within a cell. For example, proteins may act as enzymes, interact with DNA or protein, contribute to the cellular skeleton or possess some other function.

Unfortunately, it is difficult to predict the function of most gene products directly from their gene sequences. As a result, characterization of the biological function of any individual gene product, its association with disease and its pharmaceutical applications are all problems that need to be addressed even after a gene is identified.

One post-genomics field, proteomics, is attempting to bridge the knowledge gap between gene sequences and their biological functions. However, the difficulties facing proteomics are multifaceted. Unlike genes that comprise only four nucleotides and a relatively simple double helical structure, proteins are polymers that comprise different combinations of twenty different amino acids. The amino acid sequence of a protein affects the structure of the protein and hence its function. Some proteins also undergo post-translational modifications that affect their structure and biological activity.

The way in which a protein is expressed also affects the role that the protein plays within a cell. A protein may be expressed or not expressed in response to different conditions, in response to the presence of different agents, and at different levels. Where a protein is expressed within a cell and where the protein is transported after expression also impact the protein's function.

The degradation rate of a protein both affects and evidences its role within a cell. For example, short-lived proteins, i.e., proteins with a short half-life, are believed to be very important proteins in cells. It has been commented that the most important proteins will be shown to be short-lived and that most short-lived proteins will be shown to be important.

Examples of proteins that have already been shown to be short-lived include tumor suppressor p53, oncoprotein myc, cyclins, signaling protein IκB, and key biosynthetic enzymes such as ornithine decarboxylase. Their rapid turnover makes it possible for their cellular level to change promptly when synthesis is increased or reduced. Schimke, R. T. (1973) Control of enzyme levels in mammalian tissues. Advanced Enzymology, 37, 135-187.

It is believed that many proteins that turn over rapidly within cells have regulatory roles. For example, transcription factors, cell cycle regulators and metabolic enzymes are all believed to be relatively short-lived proteins.

Identifying whether a given protein is short-lived is very useful toward identifying the protein's role within the cell. Unfortunately however, analysis of whether a given protein is short-lived is currently time-consuming and labor-intensive. The most definitive form of analysis requires pulse-chase labeling cells and immunoprecipitating extracts. In vitro assay of degradation is simpler than in vivo analysis, but an in vitro assay system is difficult to establish and may not fully mimic the degradation of proteins in cells.

Identifying which proteins among all the proteins expressed by a cell are short-lived is highly desirable since it may serve to identify which proteins are the more important proteins to study. However, genome-wide functional screening and systemic characterization of cellular short-lived proteins is more complicated than analyzing the lifetime of a single known protein. Identification of short-lived proteins is more difficult because they are degraded more rapidly and tend to be present in lower quantities within the cell. Short-lived proteins are thus harder to detect, isolate and characterize. A need currently exists for a technology that allows for high throughput screening of whether proteins are short-lived.

### SUMMARY OF THE INVENTION

The present invention relates to methods, compositions and kits for detecting and characterizing short-lived proteins. Through the present invention, it is possible to perform genome-wide functional screening and systemic characterization of cellular short-lived proteins.

In one aspect of the invention, methods are provided for selecting cells based on whether the cells express a short-lived protein.

According to one embodiment of the method, the method comprises: taking a library of cells, the cells in the library expressing a fusion protein comprising a reporter protein and a protein encoded by a sequence from a cDNA library derived from a sample of cells, the sequence from the cDNA library varying within the cell library; modifying a rate of protein expression or degradation by cells in the library; and selecting a population of cells from the library of cells based on the population of cells having different reporter signal intensities than other cells in the library, the difference being indicative of the population of cells expressing shorter lived fusion proteins than the fusion proteins expressed by the other cells in the library.

According to another embodiment of the method, the method comprises: taking a library of cells, the cells in the library expressing a first reporter protein and a fusion protein comprising a second reporter protein and a protein encoded by a sequence from a cDNA library derived from a sample of cells, the sequence from the cDNA library varying within the cell library; modifying a rate of protein expression or degradation by cells in the library; and selecting a population of the cells from the library of cells based on whether the cells have a different normalized reporter signal intensity than other cells in the library, the normalized reporter signal intensity comprising a reporter signal from the fusion protein normalized relative to a reporter signal from the first reporter protein, the difference being indicative of the population of cells expressing shorter lived fusion proteins than the fusion proteins expressed by the other cells in the library.

According to yet another embodiment of the method, the method comprises: taking a library of cells, the cells in the library expressing a fusion protein comprising a reporter protein and a protein encoded by a sequence from a cDNA library derived from a sample of cells, the sequence from the cDNA library varying within the cell library; partitioning the library of cells into populations of cells based on an intensity of a reporter signal from the fusion protein such that cells partitioned into a given population have a reporter signal within a range of reporter signal intensity; modifying a rate of protein expression or degradation by cells for a given population of cells; and selecting a subpopulation of cells from the given population of cells based on whether the cells have a different reporter signal intensity than the other cells in the given population, the difference being indicative of the subpopulation of cells expressing shorter lived fusion proteins than the fusion proteins expressed by the other cells in the given population.

According to yet another embodiment of the method, the method comprises: taking a library of cells, the cells in the library expressing a first reporter protein and a fusion protein comprising a second reporter protein and a protein encoded by a sequence from a cDNA library derived from a sample of cells, the sequence from the cDNA library varying within the cell library; partitioning the library of cells into populations of cells based on an intensity of a reporter signal from the fusion protein such that cells partitioned into a given population have a reporter signal within a range of reporter signal intensity; modifying a rate of protein expression or degradation by cells for a given population of cells; and selecting a subpopulation of the cells from the population of cells based on whether the cells have a different normalized reporter signal intensity than the other cells in the population, the normalized reporter signal intensity comprising a reporter signal from the fusion protein normalized relative to a reporter signal from the first reporter protein, the difference being indicative of the subpopulation of cells expressing shorter lived fusion proteins than the fusion proteins expressed by the other cells in the given population.

According to yet another embodiment of the method, the method comprises: forming a construct library encoding a library of fusion proteins, the fusion proteins comprising a reporter protein and a protein encoded by a sequence from a cDNA library derived from a sample of cells; transducing or transfecting the construct library into cells to form a library of cells which express the library of the fusion proteins; screening the transduced or transfected cells for cells which express the fusion protein; partitioning the screened cells into populations of cells based on an intensity of a reporter signal from the fusion protein such that cells partitioned into a given population have a reporter signal within a range of reporter signal intensity; modifying a rate of protein expression or by cells in the given population; and selecting a subpopulation of the cells from the given population of cells based on whether the cells have a different reporter signal intensity than the other cells in the given population, the difference being indicative of the subpopulation of cells expressing shorter lived fusion proteins than the fusion proteins expressed by the other cells in the given population.

According to this method, the library of cells may optionally further express an internal standard protein having a different reporter signal than the reporter protein, and selecting the subpopulation of cells may optionally further comprise normalizing the reporter signal from the fusion protein using the reporter signal from the internal standard protein.

According any of the above methods, screening may be performed using a flow cytometer. In such instances, the reporter protein is preferably a protein that can be detected by the flow cytometer and used to screen the cells.

According any of the above methods, the reporter protein may be a fluorescent protein. For example, the reporter protein may be a green fluorescence protein (GFP), an enhanced green fluorescence protein (EGFP), blue fluorescence protein, yellow fluorescence protein, or a red fluorescent protein. The reporter protein may also be beta-galactosidase or luciferase.

According any of the above methods, screening and partitioning may be performed using a flow cytometer.

Also according any of the above methods, when the reporter protein is a fluorescent protein and partitioning is performed, the range of reporter signal intensity is optionally a half-log interval of fluorescence.

Also according any of the above methods, when the reporter protein is a fluorescent protein and partitioning is performed, a given population that is formed may optionally have a modal brightness that differs from another population by a factor of at least 3.

Also according any of the above methods, when the reporter protein is a fluorescent protein and partitioning is performed, partitioning may comprise partitioning the screened cells into at least 4 populations of cells where the reporter signal intensities of cells within a given population do not overlap with the reporter signal intensities of cells within another population of cells.

Also according any of the above methods, when protein expression is inhibited, selecting a subpopulation of the cells from the given population of cells may be based on cells having a reduced reporter signal intensity than the other cells in the given population.

Also according any of the above methods, when protein expression is inhibited, selecting a subpopulation of the cells from the given population of cells may be based on cells having less than half reporter signal intensity than the other cells in the given population.

Also according any of the above methods, when protein degradation is inhibited, selecting a subpopulation of the cells from the given population of cells may be based on cells having an increased reporter signal intensity than the other cells in the given population.

Also according any of the above methods, when protein degradation is inhibited, selecting a subpopulation of the cells from the given population of cells may be based on cells having more than twice the reporter signal intensity than the other cells in the given population.

Also according any of the above methods, the selected subpopulation of the cells may optionally be subjected to one or more additional rounds of selection, each round of selection comprising modifying a rate of protein expression or degradation by the cells, and selecting a further subpopulation of the cells based on whether the cells having a different reporter signal intensity than the other cells in the given population.

Also according any of the above methods, the selected subpopulation of the cells may optionally be subjected to one or more additional rounds of selection such that at least one round of selection comprises inhibiting protein expression and at least one round of selection comprises inhibiting protein degradation.

Also according any of the above methods, the selected subpopulation of cells may optionally be further selected, at least partially, by culturing cells separately and individually monitoring how the reporter signal of each cell changes in response to protein synthesis or protein degradation being inhibited.

Also according any of the above methods, the selected subpopulation of cells may optionally be further selected, at least partially, by culturing cells separately and individually monitoring how the reporter signal of each cell changes using a fluorescent plate reader.

Also according any of the above methods, the methods may optionally further comprise analyzing whether the fusion protein of the selected cells is short-lived by a pulse-chase analysis.

Also according any of the above methods, the method may optionally further comprise analyzing whether the fusion protein of the selected cells is short-lived by radiolabelling the expressed fusion protein; immunoprecipitating the expressed fusion protein with anti-GFP antisera; and analyzing the immunoprecipitate by SDS-PAGE and autoradiography.

Also according any of the above methods, the method may optionally further comprise determining the nucleic acid sequences of the fusion proteins.

Also according any of the above methods, the method may optionally further comprise determining the protein sequences of the fusion proteins.

Also according any of the above methods, the method may optionally further comprise analyzing whether the portion of the fusion protein encoded by the sequence from the cDNA library is short-lived when expressed independent of the reporter protein.

In another aspect of the invention, methods are also provided for monitoring the effects that different growth conditions have on expression of short-lived proteins

In one embodiment of the method, the method comprises: exposing samples of cells to different growth conditions; forming cDNA libraries from the sample of cells after exposure to the different growth conditions; forming a library of cells for each cDNA library, the cells in the library expressing a fusion protein comprising a reporter protein and a protein encoded by a sequence from the cDNA library derived from a sample of cells, the sequence from the cDNA library varying within the cell library; for each library of cells: identifying cells within the library that express fusion proteins that are degraded in vivo more rapidly than other fusion proteins, and characterizing fusion proteins expressed by the identified cells; and comparing which fusion proteins are characterized for each library of cells, differences in the characterized fusion proteins indicating differences in the short-lived proteins expressed by when the cells are exposed to the different agents.

In one variation of the embodiment, identifying cells within the library that express fusion proteins that are degraded in vivo more rapidly than other fusion proteins comprises modifying a rate of protein expression or degradation by the cells, and selecting a population of the cells based on whether the cells have a different reporter signal intensity than the other cells after the rate of protein expression or degradation has been modified.

In another embodiment of the method, the method comprises: exposing samples of cells to different conditions; forming cDNA libraries from the sample of cells after exposure to the different growth conditions; forming a library of cells for each cDNA library, the cells in the library expressing a fusion protein comprising a reporter protein and a protein encoded by a sequence from the cDNA library derived from a sample of cells, the sequence from the cDNA library varying within the cell library; for each library of cells: partitioning the library of cells into populations of cells based on an intensity of a reporter signal from the fusion protein such that cells partitioned into a given population have a reporter signal within a range of reporter signal intensity, modifying a rate of protein expression or degradation by the cells for a given population of cells, selecting a subpopulation of the cells from the given population of cells based on whether the cells have a different reporter signal intensity than the other cells in the given population, and characterizing fusion proteins expressed by at least a portion of the selected cells; and comparing which fusion proteins are characterized for each library of cells, differences in the characterized fusion proteins indicating differences in the short-lived proteins expressed by when the cells are exposed to the different agents.

In one variation of the embodiment, exposing the samples of cells to different conditions comprises exposing the cells to different agents such as pharmaceuticals and toxins.

In yet another aspect of the invention, a method is provided for screening for differences in short-lived proteins expressed by first and second cell samples.

In one embodiment of the method, the method comprises: forming cDNA libraries for first and second samples of cells; forming a library of cells for each cDNA library, the cells in the library expressing a fusion protein comprising a reporter protein and a protein encoded by a sequence from the cDNA library derived from a sample of cells, the sequence from the cDNA library varying within the cell library; for each library of cells: identifying cells within the library that express fusion proteins that are degraded in vivo more rapidly than other fusion proteins, and characterizing fusion proteins expressed by the identified cells; and comparing which fusion proteins are characterized for each library of cells, differences in the characterized fusion proteins indicating differences in the short-lived proteins expressed by the first and second samples cells.

In another embodiment of the method, the method comprises: forming cDNA libraries for first and second samples of cells; forming a library of cells for each cDNA library, the cells in the library expressing a fusion protein comprising a reporter protein and a protein encoded by a sequence from the cDNA library derived from a sample of cells, the sequence from the cDNA library varying within the cell library; for each library of cells: partitioning the library of cells into populations of cells based on an intensity of a reporter signal from the fusion protein such that cells partitioned into a given population have a reporter signal within a range of reporter signal intensity, modifying a rate of protein expression or degradation by the cells for a given population of cells, selecting a subpopulation of the cells based on whether the cells have a different reporter signal intensity than other cells after the rate of protein expression or degradation has been modified, and characterizing fusion proteins expressed by at least a portion of the selected cells; and comparing which fusion proteins are characterized for each library of cells, differences in the characterized fusion proteins indicating differences in the short-lived proteins expressed by the first and second samples cells.

In yet another aspect of the invention, an oligonucleotide array is provided for identifying which of a plurality of short-lived proteins are expressed in a sample. The array comprises: a substrate; and a plurality of oligonucleotide probes immobilized on a surface of the substrate such that different oligonucleotide probes are positioned in different defined regions on the surface, each of the different oligonucleotide probes comprising a binding region complimentary to a portion of a different gene encoding a short-lived protein.

The half-life of each of the short-lived proteins in its native cellular environment is preferably shorter than 12 hr, more preferably shorter than 4 hr, and most preferably shorter than 2 hr.

The oligonucleotide probes may be a DNA, RNA, PNA (peptide nucleic acid) or an equivalent thereof that is capable of binding to a portion of the RNA or DNA transcript of the gene encoding a short-lived protein. Preferably, the oligonucleotide probes are cDNA of the short-lived proteins, more preferably the sense-strand of the genes encoding the short-lived proteins, and most preferably the 3′ end of the sense-strand of the genes encoding the short-lived protein.

The length of the oligonucleotide probes is preferably between 20-100 nt, more preferably between 40-80 nt, and most preferably between 55-75 nt. The probes may be labeled with a detectable marker, such as biotin, radio-isotopes and fluorescent labels.

The density of the array may be low or high, depending on the purpose of the use of the array and/or types of short-lived proteins to be detected. The array may be a low-density one, such as one with density lower than 1000, optionally lower than 500, optionally lower than 200, optionally between 10-1000, optionally between 50-500, and optionally between 100-300. Alternatively, the array may be a high-density one, such as one with density higher than 1000, optionally higher than 10,000, optionally higher than 100,000, optionally between 1000-100,000, optionally between 5,000-50,000, and optionally between 10,000-40,000.

The diversity of the plurality of the oligonucleotide probes is optionally higher than 50, optionally higher than 500, optionally higher than 5,000, optionally between 50-5,000, optionally between 100-2,000, and optionally between 200-1,000.

The oligonucleotide array of the present invention may be used for detecting expression of many short-lived proteins simultaneously, and also for comparing expression profiles of tissues under different conditions, such as disease and normal condition.

In yet another aspect of the invention, a short-lived protein array is provided for identifying which of a plurality of agents bind to the short-lived proteins on the array. The array comprises: a substrate; and a plurality of short-lived proteins immobilized on a surface of the substrate such that different short-lived proteins are positioned in different defined regions on the surface, each of the different short-lived proteins having a halftime shorter than 24 hr in its native cellular environment.

The half-life of each of the short-lived proteins in its native cellular environment is preferably shorter than 12 hr, more preferably shorter than 4 hr, and most preferably shorter than 2 hr.

A portion of or the full-length protein of the short-lived protein may be spotted on the array covalently or non-covalently alone, or as a conjugate with another agent or a fusion with another protein.

The density of the array may be low or high, depending on the purpose of the use the array and/or types of short-lived proteins to be detected. The array may be a low-density one, such as one with density lower than 1000, optionally lower than 500, optionally lower than 200, optionally between 10-1000, optionally between 50-500, and optionally between 100-300. Alternatively, the array may be a high-density one, such as one with density higher than 1000, optionally higher than 10,000, optionally higher than 100,000, optionally between 1000-100,000, optionally between 5,000-50,000, and optionally between 10,000-40,000.

The diversity of the plurality of the short-lived proteins is optionally higher than 50, optionally higher than 500, optionally higher than 5,000, optionally between 50-5,000, optionally between 100-2,000, and optionally between 200-1,000.

The short-lived protein array of the present invention may be used for screening agents that bind to the short-lived proteins simultaneously. Such agents may be small molecules such as drugs and drug candidates, macromolecules such as DNA, RNA, and proteins, and cell or tissue lysates. For example, when the agents are a library of cellular proteins contained in a cell lysate, the cellular proteins may be labeled with a detectable marker, such as biotin, radio-isotopes and fluorescent labels. The arrays may be used for comparing binding affinity of cellular proteins towards the short-lived proteins under different conditions, such as disease and normal condition.

In yet another aspect of the invention, an antibody array is provided for identifying which of a plurality of short-lived proteins is present in a sample. The array comprises: a substrate; and a plurality of antibodies against short-lived proteins immobilized on a surface of the substrate such that different antibodies are positioned in different defined regions on the surface, each of the different short-lived proteins having a half-time shorter than 24 hr in its native cellular environment.

The antibodies may be polyclonal or monoclonal, human, non-human, chimeric, or humanized antibodies. The antibodies may be fully assembled antibodies, Fab fragments, or single-chain antibodies. The antibody may be spotted on the array covalently or non-covalently alone, or as a conjugate with another agent or a fusion with another protein.

The half-life of each of the short-lived proteins in its native cellular environment is preferably shorter than 12 hr, more preferably shorter than 4 hr, and most preferably shorter than 2 hr.

The density of the array may be low or high, depending on the purpose of the use the array and/or types of short-lived proteins to be detected. The array may be a low-density one, such as one with density lower than 1000, optionally lower than 500, optionally lower than 200, optionally between 10-1000, optionally between 50-500, and optionally between 100-300. Alternatively, the array may be a high-density one, such as one with density higher than 1000, optionally higher than 10,000, optionally higher than 100,000, optionally between 1000-100,000, optionally between 5,000-50,000, and optionally between 10,000-40,000.

The diversity of the plurality of antibodies against short-lived proteins is optionally higher than 50, optionally higher than 500, optionally higher than 5,000, optionally between 50-5,000, optionally between 100-2,000, and optionally between 200-1,000.

The antibody array of the present invention may be used for detecting short-lived proteins that bind to antibodies simultaneously. For example, when the short-lived proteins are cellular proteins, the cellular proteins may be labeled with a detectable marker, such as biotin, radio-isotopes and fluorescent labels. The arrays may be used for comparing expression profiles of short-lived proteins under different conditions, such as disease and normal condition.

In yet another aspect of the invention, a library of recombinant cells expressing a library of short-lived proteins is provided. The library of cells comprises: a library of recombinant cells capable of expressing a library of short-lived proteins from a library of heterologous expression vectors, the amino acid sequence from the library of short-lived proteins varying with the library and each of the different short-lived proteins having a half-time shorter than 24 hr in its native cellular environment.

The expression of the library of short-lived proteins may be constitutive or inducible. The expression may be controlled by a promoter heterologous to the native promoter of the short-lived protein. For example, the heterologous promoter may be a eukaryotic promoter such as insulin promoter, human cytomegalovirus (CMV) promoter and its early promoter, simian virus SV40 promoter, Rous sarcoma virus LTR promoter/enhancer, the chicken cytoplasmic β-actin promoter, and inducible promoters such as a tetracycline or its derivative inducible promoter.

The half-life of each of the short-lived proteins in its native cellular environment is preferably shorter than 12 hr, more preferably shorter than 4 hr, and most preferably shorter than 2 hr.

The diversity of the library of short-lived proteins is optionally higher than 50, optionally higher than 500, optionally higher than 5,000, optionally between 50-5,000, optionally between 100-2,000, and optionally between 200-1,000.

The recombinant cell library of the present invention may be used for screening agents that bind to the short-lived proteins simultaneously. Such agents may be small molecules such as drugs and drug candidates, macromolecules such as DNA, RNA, and proteins, and cell or tissue lysates.

It is noted that for each of the short-lived proteins identified and characterized in the present invention, a stable cell may be constructed for various applications such as in cell-based assays for screening drugs based on the short-lived proteins.

### BRIEF DESCRIPTION OF THE DRAWINGS

FIG. 1 provides a general overview of how short-lived proteins encoded by DNA from a cDNA library may be detected and characterized in a high-throughput manner according to the present invention.

FIG. 2A illustrates a process of inhibiting either protein expression or degradation and then screening for a subpopulation of cells that have a different reporter protein signal.

FIG. 2B illustrates exemplary fluorescence intensity plots for the process illustrated in FIG. 2A.

FIG. 3 illustrates a method for monitoring how degradation rates of different proteins change under different conditions.

FIG. 4 illustrates an embodiment of a method for comparing which short-lived proteins are expressed by two or more different samples of cells.

FIG. 5 shows an agarose gel analysis for verification of the sizes of cDNA inserts in the colonies randomly picked from the GFP-cDNA expression libraries. DNA marker with the arrow indicates 800 bp.

FIG. 6 illustrates FACS analysis of an EGFP-cDNA expression library transfected into 293T cells. The cells expressing EGFP are fractionated and collected into 6 subpopulations (R2, R3, R4, R5, R6, and R7) based on their fluorescence intensity.

FIG. 7 illustrates log-normal fluorescence histogram distribution from R3 and R4 populations in the presence and absence of CHX. Dashed curve represents cell populations without CHX treatment, and solid curve cell population with CHX treatment. The shade area represents sorted cells from left-shifted population. Panel A shows data from R3 population, and panel B data from R4 population.

FIG. 8 schematically illustrates a procedure for isolating and characterizing short-lived proteins described.

FIG. 9 is a table listing examples of the genes of short-lived proteins isolated in the present invention.

FIG. 10 shows Western blot analysis of three clones expressing short-lived protein with polyclonal antibodies against GFP. 1. clone5, 2. clone5 treated with CHX, 3. clone19, 4. clone19-CHX, 5.clone 26, and 6. clone 26-CHX.

FIG. 11 schematically illustrates a procedure for constructing an SH3 domain array.

FIG. 12 shows analysis of interactions between PI3 kinase and SH3 domains on the array. Panel A: The SH3 binding domain of PI3K was used as a ligand to monitor its interactions with 37 SH3 domains. The interaction of c-Src and related proteins with a PI3K was detected, which is consistent with results published in the literature. Panel B: The positive interactions were verified using pull-down assays. Panel C: The SH3 domain array was incubated with anti-GST antibody and all spotted GST-fusion proteins were shown to be present in approximately equal amounts.

### DETAILED DESCRIPTION OF THE INVENTION

Proteins that degrade more rapidly than other proteins in vivo (i.e., proteins with short half lives) are believed to be functionally significant and hence proteins whose study should be prioritized. By identifying these proteins and better understanding their function and how their expression and degradation are regulated, a myriad of therapeutic applications can be developed. For example, it may prove therapeutically advantageous to induce or inhibit expression of certain of these proteins for selected disease states. It may also prove therapeutically advantageous to develop inhibitors for certain of these proteins for selected disease states. It may also prove therapeutically advantageous for certain disease states to increase or decrease the half life of these proteins in vivo, for example by stimulating or inhibiting the regulatory pathway controlling the degradation of these proteins.

As will be described herein, the present invention provides high throughput methods that allow short-lived proteins to be identified and studied more efficiently. For example, the present invention relates to methods for identifying which proteins expressed by a given cell sample are degraded more rapidly than other proteins also expressed by the cell sample. The more rapidly degraded proteins are referred to herein as “short-lived proteins.” By understanding which proteins are short-lived, these proteins may be targeted for further study.

Expression of at least some short-lived proteins is regulated. The present invention also relates to methods for identifying short-lived proteins whose expression is affected by particular conditions. By knowing what conditions affect the expression of different short-lived proteins, therapeutic applications may be developed to induce or inhibit their expression.

The degradation rate of some proteins may also be regulated. The present invention relates to methods for identifying short-lived proteins whose degradation rate in vivo is affected by particular conditions. By knowing what conditions affect the degradation of different short-lived proteins, how protein degradation of particular short-lived proteins is regulated can be better understood. Further, therapeutic applications can be developed as a result of better understanding how degradation of these proteins is regulated and what agents influence their degradation.

Compositions and kits for use in combination with the various methods of the present invention are also provided.

Advantageously, the methods of the present invention are high-throughput methods in the sense that they can be used to perform genome-wide functional screening and systemic characterization of groups of cellular proteins as short-lived proteins. Because short-lived proteins are likely to be functionally significant, the ability to systematically identify certain proteins as being short-lived greatly assists in identifying which are the more important proteins being expressed. Given that many short-lived proteins are regulatory proteins, knowing which proteins are short-lived also helps to determine the functional significance of these proteins.

Using the technology of the present invention, functional identification of important regulatory proteins from the entire human genome is made possible in a high-throughput screening format. With this technology, human genes can be systematically screened and new genes can easily be identified from expression libraries. Because of their importance in biological function, these short-lived proteins have a great potential in drug discovery.

As will become evident by the following description of the invention, the methods of the invention advantageously allow one to differentiate and identify short-lived proteins from longer lived proteins without knowing in advance which proteins are short-lived and without knowing in advance the sequences of the various short-lived proteins that will ultimately be identified.

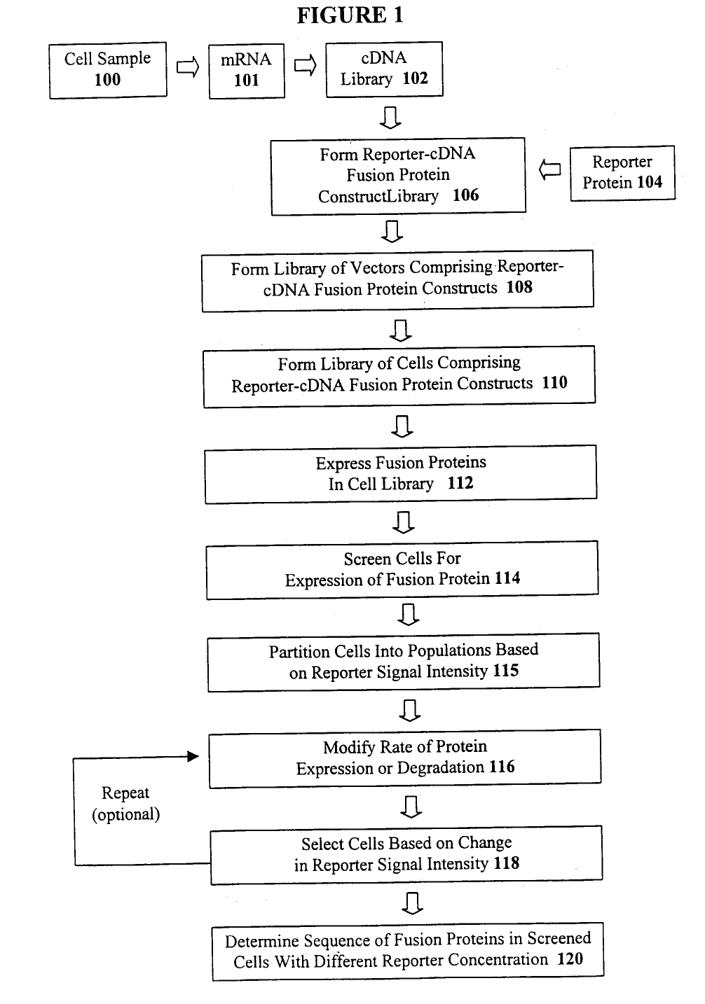

FIG. 1 provides a general overview of how short-lived proteins may be detected and characterized in a high-throughput manner according to the present invention.

As illustrated, mRNA 101 is obtained from a cell sample 100. A cDNA library 102 is then formed from the mRNA 101. The cDNA library 102 and a sequence encoding a reporter protein 104 are combined to form a construct library 106 encoding fusion proteins, each fusion protein comprising a protein encoded by a sequence from the cDNA library and the reporter protein.

A vector library 108 is formed from the construct library 106 in order to introduce the fusion protein constructs into a cell line. Introduction of the vector library may be performed by transduction or transfection, depending on the nature of the vector and the nature of the cell line.

A library of cells 110, once formed using the vector library, express the library of fusion proteins. The library of expressed fusion proteins comprise short-lived fusion proteins and a larger number of longer-lived fusion proteins. Described herein is a process for selecting cells from the library that express fusion proteins that behave as short-lived proteins over the larger group of cells that express fusion proteins that behave as longer-lived proteins.

As seen in step 112, the fusion proteins are expressed by the library of cells. The cells are then screened 114 for expression of the fusion protein based on detection of the reporter signal. The screen 114 serves to remove cells that do not exhibit a reporter signal. As a result, cells that express a fusion protein are separated from cells that either did not receive a construct or received a non-productive construct.

The reporter protein should be a protein whose expression may be detected in vivo. A variety of such proteins may be used, most commonly fluorescent proteins such as green fluorescence protein (GFP) and enhanced green fluorescence protein (EGFP) which may be readily detected and used to screen the cells by a flow cytometer.

After the cell library is screened 114, the screened cells are partitioned 115 into populations of cells where the measured reporter signal from the fusion protein in a given population is within a predetermined range. For example, if the reporter is fluorescent, the cells are grouped into populations where all the cells in a given population fluoresce within a given range of fluorescence intensity.

For a given population of cells, the rate at which protein expression or degradation occurs is then modified 116. A subpopulation of the cells is then selected 118 from the given population of cells based on those cells having different reporter signal intensities than the other cells in the given population, the difference in reporter signal intensities being indicative of the subpopulation of cells expressing shorter lived fusion proteins than the fusion proteins expressed by the other cells in the given population. The subpopulation of cells selected will typically represent a minority of the cells of the given population.

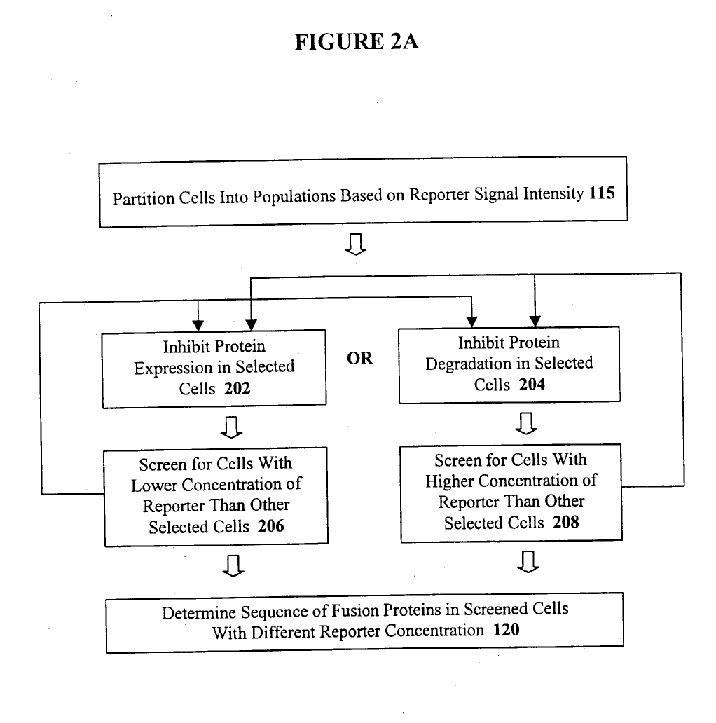

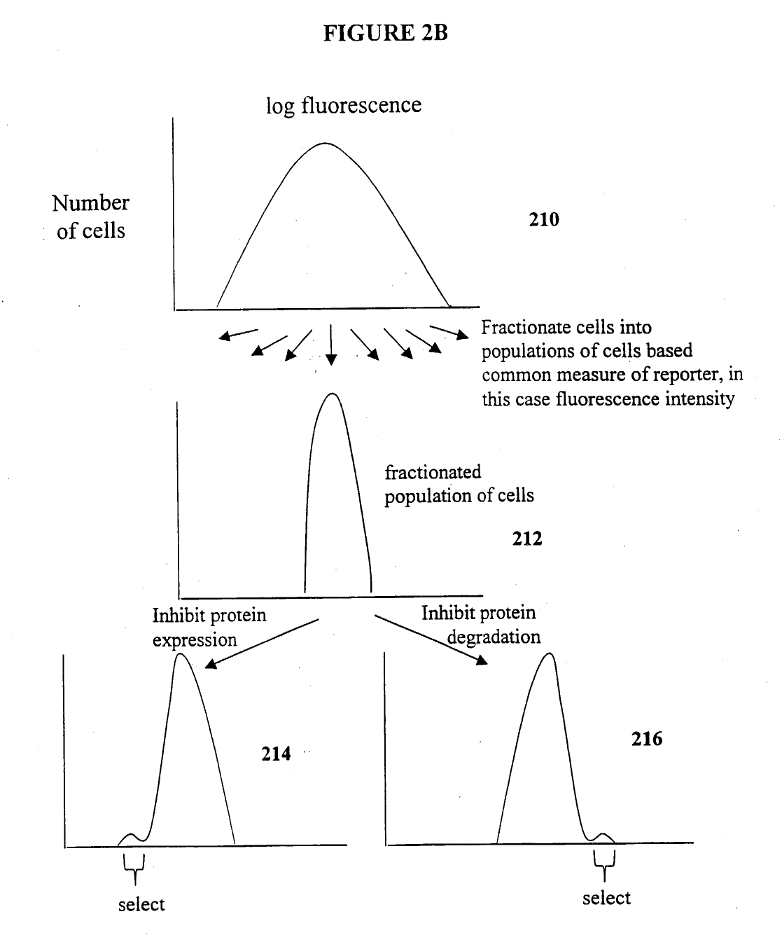

The process of partitioning the cells into populations 115, modifying the rate of protein expression or degradation 116, and selecting a subpopulation of cells based on reporter signal intensity 118 is described in more detail in regard to FIGS. 2A and 2B.

Referring to partitioning the cells into populations 115, FIG. 2B illustrates a plot of fluorescence for cells expressing fusion proteins where the reporter is fluorescent. As illustrated, the different cells have a range of fluorescence intensities 210. In order to better monitor changes in fluorescence intensities for individual cells, the cells are fractionated into populations of cells where cells in a given population are all within a narrower range of fluorescence. For example, the fluorescence plot of one fractionated population of cells 212 is shown in FIG. 2B.

Referring to the step of modifying the rate of protein expression or degradation 116 of FIG. 1, it is noted that short-lived proteins are degraded faster than other proteins. As a result, when protein expression is inhibited, the concentration of short-lived protein in the cell will decrease at a more rapid rate than longer-lived proteins because protein expression is not replacing the short-lived proteins. As a result, the reporter signal intensity in cells expressing a short-lived fusion protein will decrease more rapidly than other cells within a given population. Referring to FIG. 2A, it is possible to inhibit protein expression 202 and then select cells 206 expressing a short-lived fusion protein by selecting those cells whose reporter signal is lower than other cells in the cell population. Exemplary fluorescence intensity plots for this process are illustrated in FIG. 2B where a population of cells that initially had a common fluorescence intensity (as shown in plot 212) has separated over time into two populations where a small sub-population has a lower fluorescence intensity after protein synthesis is inhibited (as shown in plot 214).

When protein degradation is inhibited in step 116 of FIG. 1, because short-lived proteins are degraded faster than other proteins, the concentration of short-lived proteins will increase at a more rapid rate than will longer-lived proteins. As a result, the reporter signal of cells expressing a fusion protein comprising a short-lived protein within a given population will increase more rapidly than cells expressing a fusion protein comprising a longer-lived protein. Referring again to FIG. 2A, it is possible to inhibit protein degradation 204 and then select those cells 208 that express a short-lived fusion protein by selecting those cells whose reporter signal is higher than other cells in the cell population. Exemplary fluorescence intensity plots for this process are illustrated in FIG. 2B where a population of cells that initially had a common fluorescence intensity (as shown in plot 212) has separated over time into two populations where a small sub-population has a higher fluorescence intensity after protein degradation is inhibited (as shown in plot 216).

As illustrated in FIGS. 1 and 2A, the process of inhibiting either protein expression or degradation and then screening for a subpopulation of cells which have a different reporter protein signal may be performed once or repeated one or more times in order to more carefully select cells expressing short-lived fusion proteins. For example, in one variation, at least one selection is performed after inhibiting protein expression and at least one selection is performed after inhibiting protein degradation.

Optionally, the cells selected as having a different reporter signal than other cells in the population in response to protein synthesis or protein degradation being inhibited may be further evaluated prior to sequencing the fusion proteins. For example, as described herein, different cells may be cultured separately and then individually monitored for how their reporter signal changes in response to protein synthesis or protein degradation being inhibited. By monitoring the reporter signal behavior of different cells separately, it is possible to more carefully evaluate whether a given fusion protein is being degraded as would a protein with a relatively shorter half life. As a result, a more careful cell selection may be performed.

After cells believed to encode short-lived fusion proteins are finally selected, the nucleic acid and protein sequences of the fusion proteins may be determined.

Once the sequences of the fusion proteins and the cDNA encoding them are known, a variety of additional analyses may be performed. For example, database searches may be performed based on the cDNA or protein sequences in order to determine whether the cDNA sequence and/or the protein encoded by the cDNA sequence are already known. In some instances, the proteins identified by the above selection process will be novel. Even if some of the proteins are already known, their cDNA sequences may not have been known. Furthermore, the fact that these proteins are degraded more rapidly is valuable information since it indicates that these proteins may be regulatory proteins.

As can be seen from the above description, the process of the present invention allows one to screen an entire cDNA library for proteins whose difference in degradation rates evidence that these proteins are short-lived. The proteins and their cDNA need not be known prior to performing the process of the present invention or known even when performing the process. Rather, only those proteins that are likely to be short-lived proteins need to be sequenced according to the present invention.

As can also be seen, the method of the present invention allows the discovery of various valuable pieces of information that all incrementally help to fill the proteomics knowledge gap.

By being able to rapidly identify proteins as being short-lived in combination with the cDNA sequences encoding the proteins, a myriad of applications arise, some of which are described herein in further detail. For example, by determining which proteins are short-lived, arrays comprising cDNA for the short-lived proteins can be produced which allow one to rapidly monitor how expression of different short-lived proteins changes under different conditions.

The design, operation and applications for the present invention will now be described in greater detail.

1. Formation of Reporter-cDNA Fusion Protein Construct Library

In order to systematically clone all genes whose products may be short-lived, a fusion expression library is formed by combining a sequence encoding a reporter protein with a cDNA library formed from mRNAs isolated from a sample of cells. A wide variety of methods are known in the art for forming a cDNA library from mRNA isolated from a cell sample. Any of these methods may be used in the present invention.

In one embodiment, an agent such as Trizol reagent (Gibco BRL) is used to isolate total RNA from cells or a tissue sample. Oligo (dT) columns is then used to purify poly (A)⁺ RNAs. First-strand cDNA synthesis may then be primed from poly (A)⁺ RNAs by oligo dT primers. A cDNA library may then be constructed using SMART (Switching Mechanism at 5′end of RNA template) library construction technology from CLONTECH. This method simultaneously employs the two intrinsic properties of M-MLV, namely RT-reverse transcription of mRNA template and template switching activity. The technique allows two different restriction sites to be added to the anchor and oligo dT primers, to conduct directional cloning cDNAs.

Optionally, the oligo(dT) primer may include an BamHI site and an EcoRI site may be introduced into the anchor. First strand synthesis is then performed with 5-methyl dCTP, producing hemimethylated cDNA, with the unmethylated BamHI site on the linker/primer. Second-strand cDNA is generated with the unmethylated EcoRI site on the anchor as a primer, using an enzyme mixture of E. coli DNA polymerase, RNA ligase and RNase H. The double-stranded cDNA is digested with appropriate restriction enzymes to generate two different sticky ends. After size fractionation, the cDNA may be directionally cloned into expression vectors. Compared to cDNA cloned nondirectionally, libraries made according to this method are more likely to make functional fusion proteins for expression screening.

The reporter protein may be any protein that enables cells expressing the reporter protein as part of a fusion protein to be screened in vivo. The sequence encoding the reporter protein may be 3′ or 5′ relative to the sequence from the cDNA library.

In one embodiment, the reporter protein is an autofluorescent protein. A unique feature of autofluorescent proteins is their ability to be detected without any substrate or cofactor. Using an autofluorescent protein as the reporter, fluorescence associated with single cells can be analyzed by fluorescence activated cell sorting (FACS), a technology easily adapted to high throughput screening. Galbraith, D. W., Anderson, M. T. and Herzenberg, L. A. (1999) Flow cytometric analysis and FACS sorting of cells based on GFP accumulation. Methods Cell Biol, 58, 315-41. Thus, FACS can be used for analysis of the large number of human genes.

Green fluorescent protein (GFP) is an example of an autofluorescent protein. GFP from the jellyfish Aequorea victoria has been widely used to study gene expression and protein localization. Tsien, R. Y. (1998) The green fluorescent protein. Annu Rev Biochem, 67, 509-44. GFP has also been found in a variety of other organisms including Renilla.

Enhanced GFP (EGFP) is a mutant of GFP with 35-fold increase in fluorescence, which dramatically improves the detection of GFP. The fluorescence of GFP is dependent on the key sequence Ser-Tyr-Gly (amino acids 65 to 67) that undergoes spontaneous oxidation to form a cyclized chromophore. Enhanced GFP (EGFP) contains mutations of Ser to Thr at amino acid 65 and Phe to Leu at position 64, and is encoded by a gene with human-optimized codons. Cormack, B. P., Valdivia, R. H. and Falkow, S. (1996) FACS-optimized mutants of the green fluorescent protein (GFP). Gene, 173, 33-8.

A wide variety of methods are known in the art for forming a fusion protein library between a first protein (in this case the reporter protein) and sequences from the cDNA library. In one embodiment, the fusion protein libraries are constructed by fusing cDNA to the C terminus of the reporter protein, such as GFP or EGFP. Optionally, pEGFP-N1, N2, and N3 (CLONTECH) may be used to express GFP fusion proteins. pEGFP-N1, N2, and N3 are a set of vectors with three open reading frames. The vectors contain the CMV promoter, multiple cloning sites (MCS), the EGFP gene and an SV40 poly A site. The MCS with three reading frames allows genes to be cloned 5′ relative to the EGFP gene. The expression vectors also contain the SV40 origin of replication, which allows extra-chromosomal replication and facilitate recovery from cells, such as COS-7, that express the SV40 large T antigen.

2. Formation of Vector Library Comprising Reporter-cDNA Fusion Protein Constructs

A variety of different vectors may be formed to transfer the library of constructs into a cell line. These vectors may introduce the constructs into the cell line by transfection or transduction. For example, the library of constructs may be ligated into expression vectors such as pd1EGFP, pd2EGFP, and pd4EGFP which are each commercially available mammalian expression vectors that code for the fluorescence protein EGFP. These constructs are made from pEGFP-C1 with the C-terminal fusion of the degradation domain of mouse ornithine decarboxylase and demonstrated in cells with a short half-life, a range from 1 hour to 4 hours. To normalize the transfection, a second reporter construct, such as beta-galactosidase, can be co-transfected with the fluorescence protein construct under the control of the same or a different promoter.

3. Formation of Library of Cells Comprising Reporter-cDNA Fusion Protein Constructs

The library of vectors encoding the reporter-cDNA fusion proteins are then introduced into a cell line to produce a library of cells which express the reporter-cDNA fusion proteins. Preferably, the cell library formed has a diversity of at least>10⁴, more preferably>10⁵, and most preferably a diversity of at least>10⁶.

The recipient cell line of the vector library is preferably of a same genus as the sample of cells from which the cDNA library is derived. For example, a fusion protein library formed from cDNA derived from mammalian cells is preferably formed in a mammalian cell line. Similarly, a fusion protein library comprising cDNA derived from plant cells is preferably formed in a plant cell line.

In one embodiment, when the cDNA library is derived from a mammalian cells, the recipient cell line of the vector library is CHO cells or COS-7 cells. When a pd2EGFP vector is employed, it is desirable to use COS-7 cells because these cells express the SV40 large T antigen which results in high-copy extra-chromosomal replication of the pd2EGFP vector.

Once the library of cells is formed, the library is allowed to express the fusion proteins and is then screened for whether the fusion protein is being expressed. For example, when the reporter is a fluorescent protein, such as GFP or EGFP, the cells can be efficiently screened by FACS sorting. This allows one to easily separate transformed or transfected cells from untransformed or untransfected cells and cells that were transformed or transformed by non-productive constructs.

4. Sorting Cell Library Into Populations Based on Reporter Signal Intensity

The library of cells formed by transfecting or transducing a cell line with vectors encoding a library of fusion proteins will have a distribution of reporter signal intensities. For example, when the reporter is a fluorescent protein, a cell population with an approximately log-normal fluorescence histogram distribution may have a fluorescence distribution of 4 logs to the base 10.

According to the present invention, cells that are likely to encode short-lived proteins are selected by detecting changes in the cells' reporter signal intensity over time. By narrowing the distribution of reporter signal intensities within a given population of cells, it is possible to detect changes in the reporter signal intensities of individual cells within the population of cells. Therefore, prior to inhibiting protein synthesis or protein degradation, the cell library is first divided into populations, each with a distinct and narrow distribution of reporter signal intensities. Together, the populations cover the full dynamic range of the library of cells. In one variation, the cell library is divided into 2, 3, 4, 5, 6, 7, 8, 9, 10 or more populations.

When a fluorescent reporter protein is employed, FACS fractionation may be used to divide the library into separate populations where each population has a distinct and narrow fluorescence brightness distribution. Optionally, each population may be fractionated to within a half-log interval of fluorescence. This would cause each population to have a modal brightness that differs from that of an immediately adjacent population by a factor of about 3.3.

After the library is divided into separate populations with a narrower distribution of reporter signal intensities than the library, the distribution of reporter signal intensities for each population may be checked to confirm that the cells in a given population have the desired distribution of reporter signal intensities. If the population is not found to have the desired reporter signal intensity distribution, the population may be fractioned again. This process may be repeated as many times as necessary in order to produce populations of cells which each have the desired distribution of reporter signal intensities within the population.

5. Selecting Cells By Inhibiting Protein Expression and/or Protein Degradation

Once separate populations of cells are formed, each population is separately analyzed for the presence of short-lived proteins.

For a given population, a subpopulation of cells is selected based on time-dependent changes in the reporter signal intensity of the cells within the population in response to inhibiting either protein synthesis or protein degradation. This selection process may be repeated multiple times where the subpopulation of cells formed in a given round is further screened and narrowed in a later selection round. Optionally, the multiple rounds of selection include inhibiting protein synthesis and protein degradation in separate rounds. When both types of inhibition are performed in separate selections, a finer screen is accomplished.

In one embodiment, cells that have been partitioned into a population of cells having a desired distribution of reporter signal intensities are selected based on how inhibition of protein synthesis reduces the reporter signal intensity. A variety of different agents may be used to inhibit protein synthesis. Examples of such agents include, but are not limited to cycloheximide, clindamycin, azithromycin, clarithromycin and mupirocin.

When protein synthesis is reduced or blocked, short-lived proteins are more readily degraded. Hence, the signal of the reporter in the fusion protein decreases. By selecting those cells whose reporter signal decreases more rapidly than other cells, one is able to detect cells expressing a short-lived fusion protein.

In one embodiment, cells that have been partitioned into a population of cells having a desired distribution of reporter signal intensities are selected based on how inhibition of protein degradation increases the reporter signal intensity. A variety of different protein degradation inhibiters may be used. One such inhibitor is lactacystin, a specific proteasome inhibitor. Fenteany, G., Standaert, R. F., Lane, W. S., Choi, S., Corey, E. J. and Schreiber, S. L. (1995) Inhibition of proteasome activities and subunit-specific amino-terminal threonine modification by lactacystin. Science, 268, 726-731; Omura, S., Fujimoto, T., Otoguro, K., Matsuzaki, K., Moriguchi, R., Tanaka, H. and Sasaki, Y. (1991) Lactacystin, a novel microbial metabolite, induces neuritogenesis of neuroblastoma cells. J Antibiot (Tokyo), 44, 113-6.

When degradation of short-lived proteins is inhibited, the concentration of short-lived proteins increases within the cell. This results in the signal of the reporter in the fusion protein increasing. By selecting those cells whose reporter signal increases more rapidly than other cells, one is able to detect cells expressing a fusion protein comprising a short-lived protein.

Exposure to agents that inhibit protein synthesis and protein degradation should be controlled so that live cells may be recovered and further processed. Hence, exposure to inhibitors should be limited to durations that are consistent with survival. Also, it is recognized that prolonged exposure could induce a secondary cellular response that produces alterations in signal intensity from causes other than protein turnover. This could result in a false-positive background. As discussed herein, a second reporter protein may be used as an internal standard to counter these potential alterations in reporter signal intensity.

The duration desirable for inhibiting protein synthesis or protein degradation is dependent upon how great a change in the signal intensity of the reporter is to be detected. It is also dependent upon the desired maximum half life of the proteins to be detected. For example, cells may be selected which show at least a 2×, 4×, 6×, or 8×change in reporter signal intensity. This change in reporter signal intensity may occur over varying lengths of time, such as within 1 hour, 2 hours, 3 hours, etc. In the case of inhibiting protein synthesis, the half life of a protein would be expected to equal the time required for the reporter signal intensity associated with the protein to decrease by 50%, assuming no pharmacological lag. Hence, a protein with 2 times less reporter signal intensity after an hour would be expected to have a half life of about 1 hour. Similarly, a protein with 4 times less reporter signal intensity after two hours and a protein with 8 times less reporter signal intensity after three hours would both be expected to have a half life of about 1 hour, assuming no pharmacological lag.

As described above, prior to inhibiting protein synthesis or protein degradation, the cell library is divided into populations, each with a distinct and narrow distribution of reporter signal intensities. When a fluorescent reporter protein is used, each population will have a distinct and narrow fluorescence brightness distribution. Together, the populations cover the full dynamic range of the library of cells.

Each population is subjected individually to one or more protein synthesis or protein degradation inhibitor selections. For each selection, cells are selected from the population which by their reporter signal intensity behave differently than a main portion of the population. For example, cells may be selected from the population which fall outside of the mean reporter signal intensity for the population by a factor of two, three, four, five, ten or more.

The subpopulation of cells selected after each round of selection is expected to constitute a very small fraction of the cell population prior to the selection.

Cells that are selected during each selection round are washed free of the protein synthesis or protein degradation inhibitor and allowed to regenerate through cell division in culture. After regeneration, the cells may be subjected to further rounds of selection.

Gene recovery and sequence analysis may be performed on cells selected after one or more rounds of selection in order to identify the fusion protein expressed by the selected cells. Gene recovery and sequence analysis may be performed by any of a large number of well-known techniques.

6. Optional Further Selection of Cells

The selection process described in Section 5 serves to enrich the percentage of cells in the resulting population of selected cells that encode a short-lived protein. Optionally, further selection may be performed where individual clones of the selected cells are further analyzed for whether they encode a short-lived protein.

According to this variation, the selected cells are separated such that single cells are seeded into wells of microtiter plates and allowed to grow, preferably to at least 10⁴ cells per well. The wells may then be treated with a protein synthesis or protein degradation inhibitor. Afterward, the individual wells are scanned to assess time-dependent changes in the reporter signal. Wells exhibiting time-dependent changes indicative of the cells expressing short-lived proteins may be marked and the cells contained therein recovered. Gene recovery and sequence analysis may then be performed on the recovered cells.

This additional selection of individual clones can be carried out manually with the aid of a fluorescent plate reader. Higher throughput may be desirable or even necessary if large numbers of cells need to be screened, for example, because the selection process yields a small population of desired cells. High throughput screening may be carried out using a Cellomics ArrayScan Kinetics HCS Workstation (Cellomics, Pittsburgh).

7. Validation of Selection Process

In order to validate the specificity of the selection process, cells that are selected may be analyzed using conventional methods to evaluate protein lability. For example, pulse-chase analysis may be performed to confirm whether the fusion protein expressed by the selected cells are short-lived. When GFP is used as the reporter protein, this validation may be performed by immunoprecipitating the labeled fusion protein with anti-GFP antisera, followed by SDS-PAGE and autoradiography.

8. Internal Standard For Monitoring Selection Efficiency

Stochastic cellular processes can induce the fluorescence signals of some cells to change over time. For example, changes in cell shape, cell cycle position, or intracellular redistribution of a fusion protein can all cause the fluorescent signal of a cell to change. When selecting cells based on a change in fluorescence, false positives may be selected if the fluorescence signals of those cells change in a manner that causes the cells to be mistakenly selected as expressing short-lived fusion proteins.

Multiple rounds of population-based selections using FACS will serve to eliminate false positives misidentified as a result of such random fluctuations. False positive selections will also be eliminated in subsequent, more individualized screens.

It is nevertheless desirable to reduce the frequency with which false positives are at least initially selected. This can be achieved by using an internal standard whose signal also varies as a result of these stochastic cellular processes. As a result, by normalizing the reporter relative to the internal standard, a normalized reporter value can be determined that is more reliably indicative of the expression of the reporter.

For example, cells may be transformed or transfected so they express a fusion protein comprising the first reporter protein and a second reporter protein, such as beta-galactosidase, that has a different emission wavelength than the first reporter protein. This allows expression of the first reporter protein and the second reporter protein to be independently monitored. It also allows the signal from the first reporter protein for each cell to be normalized relative to the second reporter protein. The normalized reporter signal for a given cell should be less effected by the stochastic cellular processes of that cell. Hence, basing selection upon the normalized reporter signals for each cell should reduce the frequency of false positives.

The second reporter protein may be introduced into cells by any manner and by any vehicle. For example, the second reporter protein may also be introduced into the cell by transformation or transfection and may be introduced before, after, or with the introduction of the vector encoding the fusion protein.

In one embodiment, the vector library comprising the first reporter—cDNA fusion protein constructs further encodes the second reporter protein. Hence, initial selection of cells for whether the cells received a vector from the vector library may be based either upon the first reporter protein or the second reporter protein.

Optionally, cells may be added to each population which express a known short-lived protein as a benchmark. These benchmark cells for each population should have a brightness mode that is close to that of its related population. The benchmark cells may be added in known concentrations, for example in numbers that constitute 1:100, 1:1000 or 1:10,000 of total cells. The benchmark cells may also be marked with a benchmark reporter protein, such as beta-galactosidase. Since other cells in the population will not express the benchmark reporter protein, the effectiveness of the present invention to enrich the concentration of short-lived proteins relative to the initial cell library can be monitored by measuring the frequency of this marker.

9. Characterizing Sequence From cDNA Library in Selected Cells

After selecting cells whose reporter signal behavior indicates that the fusion protein is short-lived, the sequences encoding the fusion protein may be analyzed. Specifically, the selected cells may be pooled and extra-chromosomal DNA extracted and transfected into E. coli. It is noted that other methods may be used to recover the gene inserts. For example, the gene inserts can be recovered through PCR, using flanking sequences from the vector used to introduce the sequence encoding the fusion protein as a primer.

The E. coli library produced by transfecting the extra-chromosomal DNA may then be used to obtain DNA sequence information. Individual bacterial cells may be isolated and cultured in commercially available 384-well high-density culture plates. Each individual culture plate may be bar-coded where individual clones are assigned a particular code. This allows the cell lines to be readily retrieved for further analysis. The barcode system may be implemented throughout the entire process.

E. coli cells in replica plates are diluted and used for DNA amplification in an appropriate 384-well PCR plate. After PCR amplification, the DNA fragments can be used for, direct sequencing. A DNA sequence database may be established based on the sequence information. The DNA sequence and putative translated protein sequence can then be examined and compared with existing DNA sequence database using The National Center for Biotechnology Information (NCBI) and by using the BLAST program run by NCBI, or by The Protein Extraction Description and Analysis Tool (PDANT) program. Genes identified that are of interest may be readily retrieved from the original cell clones based on their barcodes.

10. Confirmation of Whether Isolated Proteins Are Short-Lived in Native Form

Once the DNA and protein sequences of the fusion proteins are identified, further analysis may be performed to evaluate whether the portion of the fusion protein encoded by the sequence from the cDNA library is short-lived in its native form, that is, when expressed free of the reporter protein. Testing of the lability of the native form of the protein screened via the above process may be performed by standard methods, such as pulse-chase analysis, which are known in the art.

11. Monitoring Changes in Degradation Rate of Proteins Under Different Conditions

It is noted that the degradation rate of a given protein is itself subject to regulation. Hence, different proteins may be short-lived under certain cellular conditions and less labile under other conditions. For instance, IκB, the inhibitor of NFκB, forms a complex with NFκB and inhibits NFκB activity. When the pathway is triggered by TNF or IL-1, a cascade of kinases in the NFκB pathway is activated, which results in phosphorylation and degradation of IκB. NFκB is released from the complex and translocates from the cytoplasm to nucleus to mediate transcriptional induction of a number of genes whose products are very important to immunity and inflammatory responses.

A need thus exists for methodology that allows one to monitor how degradation rates of different proteins change under different conditions.

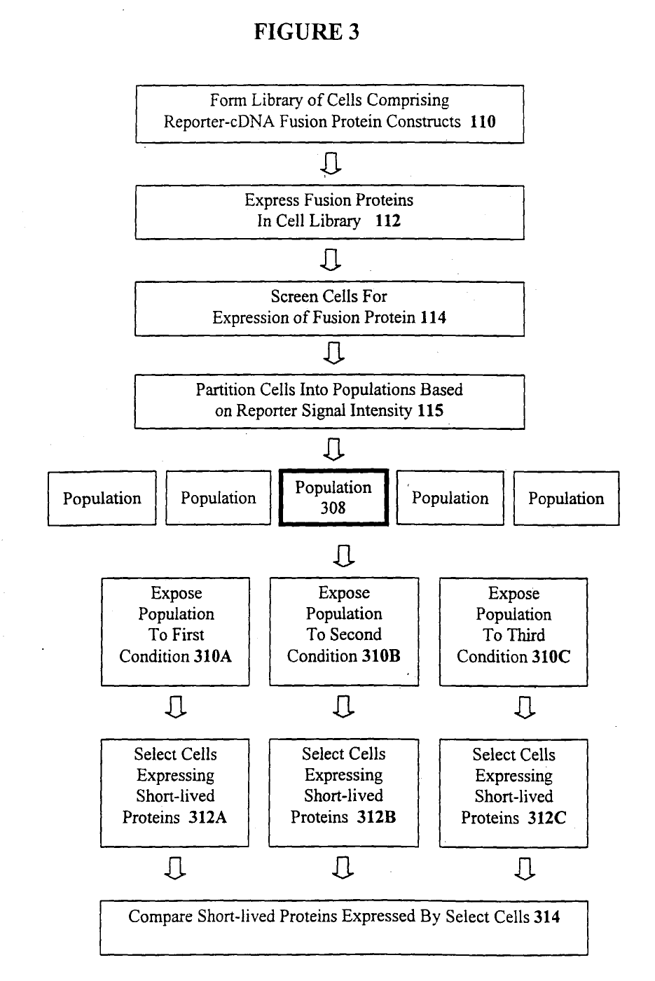

FIG. 3 illustrates a method for monitoring how degradation rates of different proteins change under different conditions. According to this variation, a library of cells expressing a fusion protein library is formed 110, screened 114 and partitioned 115 according to the present invention.

One or more of the partitioned populations of cells 308 is then grown under different conditions 310A-310C which may serve to regulate protein degradation. These different conditions may include cell cycle position, inducing conditions or other factors. For example, the different conditions may include exposing the cells to a library of agents that may affect regulation of the degradation process.

Those cells that are found to have a reporter signal behavior indicative of a fusion protein being degraded as a short-lived protein are selected 312A-312C. The selection process may comprise the one or more selection rounds and other selection processes described above.

The fusion proteins expressed by the selected populations of cells 312A-312C are then compared 314. By seeing which fusion proteins are expressed by the same population of cells 308, it is possible to determine how the different conditions influence protein degradation.

By comparing which proteins are degraded by the cells under different growth conditions and when exposed to different agents, the process of how the degradation of certain proteins is regulated can be elucidated. For example, by determining that a given protein is labile within a cell in the presence of a given agent but is otherwise a stable protein, one is able to begin to deduce how that protein is regulated. This information could lead to the identification and development of therapeutic agents that either reduce or increase the half life of selected proteins by knowing how to control the degradation regulatory pathway associated with that protein.

In some instances, conditions may affect the protein degradation of a group of proteins. By determining groups of proteins that appear to have their degradation rate linked in some way, regulatory pathways can be deduced. For example, the fact that administering an agent affects the degradation of a group of proteins may indicate that the agent is either inhibiting or inducing a given pathway. This allows the proteins involved in that pathway to be identified. By finding agents that inhibit different subgroups of proteins, the pathway may be further elucidated.

Being able to determine whether a given agent affects the degradation rate of more than one protein is very useful in designing therapeutics. For example, the fact that a given agent affects the degradation rate of multiple proteins may signal that that agent is not sufficiently selective and may cause undesirable side affects. The fact that a given agent affects the degradation rate of multiple proteins may also signal that that protein is not an attractive target for regulating a given pathway.

12. Comparing Short-lived Protein Expression Across Different Samples

In Section 11, it was noted that the degradation rate of a given protein may be affected by the conditions under which the cells are grown. In that instance, a cDNA library isolated from a single sample is tested under different conditions.

This section describes how to compare which short-lived proteins are expressed by different cell samples. When the protein expression of normal cells and diseased cells are compared, it may be found that different short-lived proteins are either expressed or not expressed by the diseased cells. For example, the diseased cells may comprise a genetic abnormality relative to the normal cells. By comparing which short-lived proteins are expressed by normal and diseased cells, it may be possible to identify one or more short-lived proteins whose expression or non-expression account for the diseased cells being abnormal. Treatments may then be directed to these identified short-lived proteins.

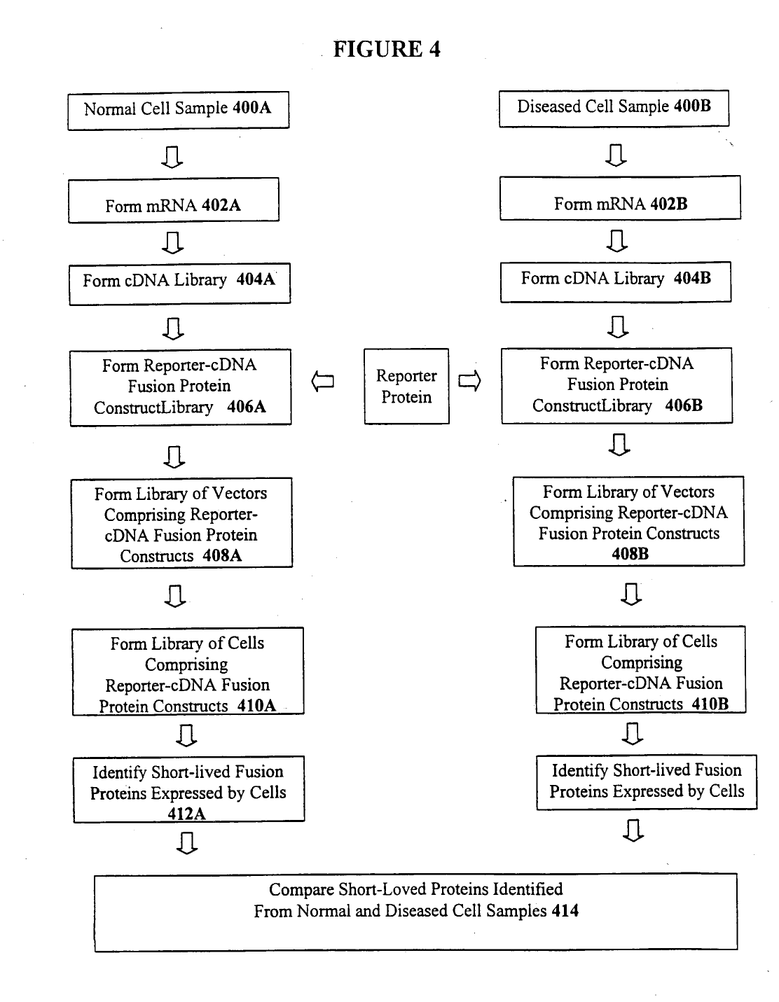

FIG. 4 illustrates an embodiment of a method for comparing which short-lived proteins are expressed by two or more different samples of cells. In FIG. 4, a normal 400A and diseased 400B sample of cells are shown. mRNA libraries 402A, 402B and then cDNA libraries 404A, 404B are formed for the cell samples 400A, 400B. Libraries of constructs 406A, 406B, libraries of vectors 408A, 408B, and then libraries of cells 410A, 410B are formed based on each cDNA library. The resulting libraries of cells are then each processed as set forth in FIG. 1 in order to identify short-lived fusion proteins expressed by each library of cells 412A, 412B. By comparing 414 which short-lived fusion proteins are expressed by each library of cells 410A, 410B, it is possible to detect differences between the libraries and hence differences between the short-lived proteins expressed by the two or more different samples of cells 400A, 400B.

13. Method for Altering Degradation Rate For Short-Lived Proteins

Proteins differ widely in their lability, ranging from entirely stable to half-lives that measure minutes. In some cases, rapidly degraded proteins have been shown to contain an identifiable “degradation domain.” Removal of this degradation domain makes such proteins stable and appending this domain to a stable protein changes its stability dramatically. Such a degradation domain has been identified in a number of short-lived proteins, such as the C terminus of mouse ODC. (Li, X., Stebbins, B., Hoffman, L., Pratt, G., Rechsteiner, M. and Coffino, P. (1996) The N Terminus of Antizyme Promotes Degradation of Heterologous Proteins. The Journal of Biological Chemistry, 271, 4441-4446; Loetscher, P., Pratt, G. and Rechsteiner, M. (1991) The C Terminus of Mouse Ornithine Decarboxylase Confers Rapid Degradation on Dihydrofolate Reductase. The Journal of Biological Chemistry, 266, 11213-11220) and the destruction box of cyclins (Glotzer, M., Murray, A. W. and Kirschner, M. W. (1991) Cyclin is Degraded by the Ubiquitin Pathway. Nature, 349, 132-138).

In some cases, the signal is a primary sequence such as the PEST sequence. Rechsteiner, M. and Rogers, S. W. (1996) PEST Sequences and Regulation by Proteolysis. Trends in Biochemical Sciences, 21, 267-271; Rogers, S., Wells, R. and Rechsteiner, M. (1986) Amino Acid Sequences Common to Rapidly Degraded Proteins: The PEST Hypothesis. Science, 234, 364-368. However, the structural features of such degradation domains are not sufficiently uniform as to provide a reliable guide to identifying the general class of labile proteins that interests us here. The major neutral protease responsible for degradation of labile regulatory proteins is the proteasome. Zwickl, P., Voges, D. and Baumeister, W. (1999) The Proteasome: A Macromolecular Assembly Designed for Controlled Proteolysis. Philos Trans R Soc Lond B Biol Sci, 354, 1501-11.

Prior to degradation, most short-lived proteins are covalently coupled to multiple copies of the 76 amino acid protein ubiquitin, a reaction catalyzed by a series of enzymes. Ciechanover, A. and Schwartz, A. L. (1998) The Ubiquitin-Proteasome Pathway: The Complexity and Myriad Functions of Proteins Death. Proc Natl Acad Sci USA, 95, 2727-30. These ubiquitinated proteins are recognized by 26S proteasome and degraded within its hollow interior. This system of regulated degradation is central to such processes as cell cycle progression, gene transcription and processing of antigens. A few proteins have been found to be exceptional. Verma, R. and Deshaies, R. J. (2000) A Proteasome Howdunit: The Case of The Missing Signal. Cell, 101, 341-4. Like ornithine decarboxylase, they do not require ubiquitin modification for degradation by the proteasome.

A desirable utility of being able to rapidly and efficiently determine the sequence of a large number of different short-lived proteins is the prospect of identifying additional degradation domains. By knowing what domains affect recognition within the cell that a protein should be degraded, it is then possible to reengineer proteins either to increase or decrease their rate of degradation in vivo.

A significant problem in the art relates to the rate at which therapeutic proteins administered to the body are cleared. With enhanced knowledge regarding how protein degradation is regulated, for example, by better understanding what are the degradation domains of proteins, it is possible to modify the degradation domains of therapeutic proteins so that these proteins have longer half lives in the body when administered.

14. Arrays Derived from Short-Lived Proteins

1) Oligonucleotide Array

The present invention also provides an oligonucleotide array which can be used for identifying which of a plurality of short-lived proteins are expressed in a sample. The oligonucleotide array comprises: a substrate; and a plurality of oligonucleotide probes immobilized on a surface of the substrate such that different oligonucleotide probes are positioned in different defined regions on the surface, each of the different oligonucleotide probes comprising a binding region complimentary to a portion of a different gene encoding a short-lived protein.

The half-life of each of the short-lived proteins in its native cellular environment is preferably shorter than 12 hr, more preferably shorter than 4 hr, and most preferably shorter than 2 hr.

The oligonucleotide probes may be a DNA, RNA, PNA (peptide nucleic acid) or an equivalent thereof that is capable of binding to a portion of the RNA or DNA transcript of the gene encoding a short-lived protein. Preferably, the oligonucleotide probes are cDNA of the short-lived proteins, more preferably the sense-strand of the genes encoding the short-lived proteins, and most preferably the 3′ end of the sense-strand of the genes encoding the short-lived protein.

The length of the oligonucleotide probes is preferably between 20-100 nt, more preferably between 40-80 nt, and most preferably between 55-75 nt. The probes may be labeled with a detectable marker, such as biotin, radio-isotopes and fluorescent labels.

The density of the array may be low or high, depending on the purpose of the use the array and/or types of short-lived proteins to be detected. The array may be a low-density one, such as one with density lower than 1000, optionally lower than 500, optionally lower than 200, optionally between 10-1000, optionally between 50-500, and optionally between 100-300. Alternatively, the array may be a high-density one, such as one with density higher than 1000, optionally higher than 10,000, optionally higher than 100,000, optionally between 1000-100,000, optionally between 5,000-50,000, and optionally between 10,000-40,000.

The diversity of the plurality of the oligonucleotide probes is optionally higher than 50, optionally higher than 500, optionally higher than 5,000, optionally between 50-5,000, optionally between 100-2,000, and optionally between 200-1,000.

The oligonucleotide array can be used for identifying transcripts of genes encoding short-lived proteins, such as regulatory proteins. Lability is a common property of regulatory proteins because it is intrinsic to their role; lability allows the level of the regulator to change quickly in response to changes in production-changes that usually depend on altered gene transcription. We believe that the transcripts from most of the genes should be highly regulated; and it is informative to array these genes and to examine changes in their transcripts under different physiological conditions. This form of analysis can establish the linkage between these regulatory proteins and gene expression patterns. It is likely that some of these genes are functionally altered in certain disease processes. These alterations in gene expression can easily be assessed by comparing the gene expression profiles of normal and diseased tissues using the arrays of the present invention. This information should provide a significant advantage in the application of gene expression data to the development of molecular diagnostics.

For example, low-density membrane-based oligonucleotide arrays can be constructed for studying the expression of a specific group of gene, e.g., a few hundred short-lived protein genes. These arrays can be developed and produced using methods for constructing low-density oligonucleotide array known in the art. A 70-bp region from the coding sequence of each short-lived protein can be used based on minimal homology to other transcripts from the human genome. The 70-bp length should be almost as sensitive as full-length cDNA products and yet with greatly reduced cross-homology to other genes. Using only the sense-strand of the 70-bp region further reduces the chances of nonspecific hybridization to the antisense strand, which would be present in PCR-amplified cDNA products. The oligonucleotide probe can be placed as far as possible towards the 3′ end of the coding sequence because this region is more likely to be synthesized in the cDNA synthesis reaction used to generate the DNA for array hybridizations.

The oligonucleotide probes can be spotted on positively charged nylon membranes in duplicates, together with housekeeping genes for normalization purposes. Biotinylated cDNA can be generated from total RNA isolated from the tissues or cells under investigation and hybridized to the arrays using standard hybridization conditions. Detection of the bound cDNA can be achieved using Streptavidin-HRP conjugates and chemiluminescence substrates on an imaging system (e.g., an Alpha Innotech imaging system). Images can be acquired and analyzed using Alpha Innotech's AlphaEaseFC software; all further analyses, such as background subtraction; normalization, and graphical display, can be performed in Microsoft Excel using customized Macros.

2) Short-Lived Protein Array

The present invention also provides a short-lived protein array which can be used for identifying which of a plurality of agents bind to the short-lived proteins on the array. The protein array comprises: a substrate; and a plurality of short-lived proteins immobilized on a surface of the substrate such that different short-lived proteins are positioned in different defined regions on the surface, each of the different short-lived proteins having a half-time shorter than 24 hr in its native cellular environment.

The half-life of each of the short-lived proteins in its native cellular environment is preferably shorter than 12 hr, more preferably shorter than 4 hr, and most preferably shorter than 2 hr.

A portion of or the full-length protein of the short-lived protein may be spotted on the array covalently or non-covalently alone, or as a conjugate with another agent or a fusion with another protein.

The density of the array may be low or high, depending on the purpose of the use the array and/or types of short-lived proteins to be detected. The array may be a low-density one, such as one with density lower than 1000, optionally lower than 500, optionally lower than 200, optionally between 10-1000, optionally between 50-500, and optionally between 100-300. Alternatively, the array may be a high-density one, such as one with density higher than 1000, optionally higher than 10,000, optionally higher than 100,000, optionally between 1000-100,000, optionally between 5,000-50,000, and optionally between 10,000-40,000.

The diversity of the plurality of the short-lived proteins is optionally higher than 50, optionally higher than 500, optionally higher than 5,000, optionally between 50-5,000, optionally between 100-2,000, and optionally between 200-1,000.

The short-lived protein array of the present invention can be used for screening agents that bind to the short-lived proteins simultaneously. Many short-lived proteins have partners that are involved in the regulation of the short-lived proteins' function or degradation. To identify such partners or profile their binding activities, an array of short-lived proteins can be a useful tool. The short-lived proteins are expressed and purified, for example as recombinant GST-short-lived fusion proteins; and then immobilized on membranes according to our established protein array technology.

The protein array of the present invention can be used for analyzing interactions of short-lived proteins with a single (known) protein or is a mixture of proteins (e.g., cell lysate). For example, when the test sample is a single known protein, the test protein can be expressed as a tag fusion protein or directly used as a probe (if its specific antibody is available). After incubation with the pre-spotted array of short-lived proteins, binding can be detected using the antibody against the protein or the fused tag. The short-lived proteins that interact with the test protein can then be identified. If the test sample is a cell lysate, the cellular proteins in the lysate can be biotinylated using a commercial labeling system (Pierce). The labeled proteins can be incubated with the protein array, and interactions between cellular proteins and short-lived proteins can be detected with Streptavidin-HRP conjugates.

The arrays may be used for comparing binding affinity of cellular proteins towards the short-lived proteins under different conditions, such as disease and normal condition. Through comparison of two different samples, the differences in interaction patterns with short-lived proteins can be determined. This comparison should provide clues about whether these two samples interact differently with the short-lived proteins. The bound protein can be further characterized by using mass spectrometry analysis, or by using the targeted short-lived protein as a probe to screen an expression library for the bound protein.

3) Antibody Array

The present invention also provides an antibody array which can be used for identifying which of a plurality of short-lived proteins is present in a sample. The array comprises: a substrate; and a plurality of antibodies against short-lived proteins immobilized on a surface of the substrate such that different antibodies are positioned in different defined regions on the surface, each of the different short-lived proteins having a half-time shorter than 24 hr in its native cellular environment.

The antibodies may be polyclonal or monoclonal, human, non-human, chimeric, or humanized antibodies. The antibodies may be fully assembled antibodies, Fab fragments, or single-chain antibodies. The antibody may be spotted on the array covalently or non-covalently alone, or as a conjugate with another agent or a fusion with another protein.

The half-life of each of the short-lived proteins in its native cellular environment is preferably shorter than 12 hr, more preferably shorter than 4 hr, and most preferably shorter than 2 hr.

The density of the array may be low or high, depending on the purpose of the use the array and/or types of short-lived proteins to be detected. The array may be a low-density one, such as one with density lower than 1000, optionally lower than 500, optionally lower than 200, optionally between 10-1000, optionally between 50-500, and optionally between 100-300. Alternatively, the array may be a high-density one, such as one with density higher than 1000, optionally higher than 10,000, optionally higher than 1100,000, optionally between 1000-100,000, optionally between 5,000-50,000, and optionally between 10,000-40,000.

The diversity of the plurality of antibodies against short-lived proteins is optionally higher than 50, optionally higher than 500, optionally higher than 5,000, optionally between 50-5,000, optionally between 100-2,000, and optionally between 200-1,000.

The antibody array of the present invention may be used for detecting short-lived proteins that bind to antibodies simultaneously. Just as they change the levels of their gene transcripts, short-lived proteins change the amount of proteins under different cellular conditions (e.g. cyclins during the cell cycle). Many short-lived proteins are specifically associated with certain types of tissues, and the levels of those proteins change dramatically among specific cells. This is because short-lived proteins perform specific functions in the host cells. The antibody array can be used to uncover the mechanisms of gene expression regulation, identify potential new targets for drug development (e.g., in the areas of cancer, immune regulation, and diabetes), and explore the applications of these proteins in clinical diagnosis. Profiling the short-lived proteins themselves—an effective way to discover differences—should be a significant step towards achieving these goals.

The antibody array of the present invention can also be used to directly monitor changes in the levels of short-lived proteins. Qualitative analysis can be performed simply by comparing the signals obtained with control and test arrays.

The antibodies against short-lived proteins can be immobilized on an array membrane by using methods known in the art. Samples used for antibody array analysis can be biotinylated with Pierce's reagent according to their provided procedure. The cellular proteins can be also labeled with fluorescence. Biotinylation may be used for membrane-based arrays, while fluorescence labeling may be used for glass arrays. After a sample is incubated with an array membrane, the bound proteins can be detected using Streptavidin-HRP conjugates and chemiluminescence substrates. When two samples are analyzed and compared in this way, the differences in levels of short-lived proteins can be identified.

Alternatively, the tested sample can be used directly for detection without biotinylation, which would eliminate any structural changes caused by the modification. As an additional advantage, users can perform array analyses of the samples without additional labeling. When this approach is used, two sets of antibodies against the short-lived proteins can be made. These two sets of antibodies can target different epitopes of the proteins, at the N- and C-termini. One set of antibodies is immobilized on a membrane for capturing short-lived proteins, and the second set is biotinylated for detection of the captured proteins. After immobilization, the antibodies arrayed on the membrane are incubated with a tested sample to profile the amounts of short-lived proteins. The specific short-lived proteins will be captured by the immobilized antibodies, and the bound proteins can then be detected using the biotinylated second set of antibodies, followed by Streptavidin-HRP conjugates and chemiluminescence substrates.

15. Compositions and Kits for Use in the Methods of the Present Invention

A wide variety of compositions and kits may be designed for use in combination with the various methods of the present invention. Various examples of these compositions, such as reporter—cDNA fusion protein construct libraries 106, vectors comprising the library of reporter—cDNA fusion protein constructs 108, and library of cells expressing the library of reporter—cDNA fusion proteins 110 have already been described herein.

In one embodiment, a library of recombinant cells expressing a library of short-lived proteins is provided. The library of cells comprises: a library of recombinant cells capable of expressing a library of short-lived proteins from a library of heterologous expression vectors, the amino acid sequence from the library of short-lived proteins varying with the library and each of the different short-lived proteins having a half-time shorter than 24 hr in its native cellular environment.

The expression of the library of short-lived proteins may be constitutive or inducible. The expression may be controlled by a promoter heterologous to the native promoter of the short-lived protein. For example, the heterologous promoter may be a eukaryotic promoter such as insulin promoter, human cytomegalovirus (CMV) promoter and its early promoter, simian virus SV40 promoter, Rous sarcoma virus LTR promoter/enhancer, the chicken cytoplasmic β-actin promoter, and inducible promoters such as a tetracycline or its derivative inducible promoter.

The half-life of each of the short-lived proteins in its native cellular environment is preferably shorter than 12 hr, more preferably shorter than 4 hr, and most preferably shorter than 2 hr.

The diversity of the library of short-lived proteins is optionally higher than 50, optionally higher than 500, optionally higher than 5,000, optionally between 50-5,000, optionally between 100-2,000, and optionally between 200-1,000.

The recombinant cell library of the present invention may be used for screening agents that bind to the short-lived proteins simultaneously. Such agents may be small molecules such as drugs and drug candidates, macromolecules such as DNA, RNA, and proteins, and cell or tissue lysates.

It is noted that for each of the short-lived proteins identified and characterized in the present invention, a stable cell may be constructed for various applications such as in cell-based assays for screening drugs based on the short-lived proteins.

It is noted that a variety of kits may be formed which may be used to construct these various compositions or which may be used in combination with these various compositions for performing aspects of the present invention. Several of these kits are described herein. Others will be well understood by one of ordinary skill in the art.

### EXAMPLE

1. Construction of a GFP-cDNA Expression Library

Messenger RNAs from brain, liver, and Hela cell line (Clontech) were used as templates for cDNA synthesis using a cDNA synthesis kit from Stratagene according to the manufacturer's procedure, with some modifications. First-strand cDNA was synthesized using an oligo(dT) primer-linker containing an Xho I restriction site and with StrataScript reverse transcriptase. Synthesis was performed in the presence of 5-methyl dCTP, resulting in hemimethylated cDNA, which prevents endogenous cutting within the cDNA during cloning. Second-strand cDNA was synthesized using E. coli DNA polymerase and RNase H. EcoRI adapters containing EcoRI cohesive ends were introduced into the double-stranded cDNA, which were then digested with XhoI. The cDNAs contained two different sticky ends: 5′ EcoRI and 3′ XhoI. The cDNAs were separated on a 1 percent seaPlaque GTG agarose gel in order to collect those larger than 800 bp. After extracting cDNAs from the agarose gel with AgarACE-agarose-digesting enzyme followed by ethanol precipitation, we directionally cloned the cDNAs into EGFP-C1/2/3 expression vectors with three open reading frames (Clontech). The vectors were modified within the multiple cloning sites in order to be compatible with the cDNA orientation. With this modification, cDNA were constructed in the library to the C-terminus of EGFP. Since the expression vectors contain the SV40 origin of replication, cDNA clones that show positive in the screening can be easily recovered from cell lines that express the SV40 large T antigen (e.g., 293T).

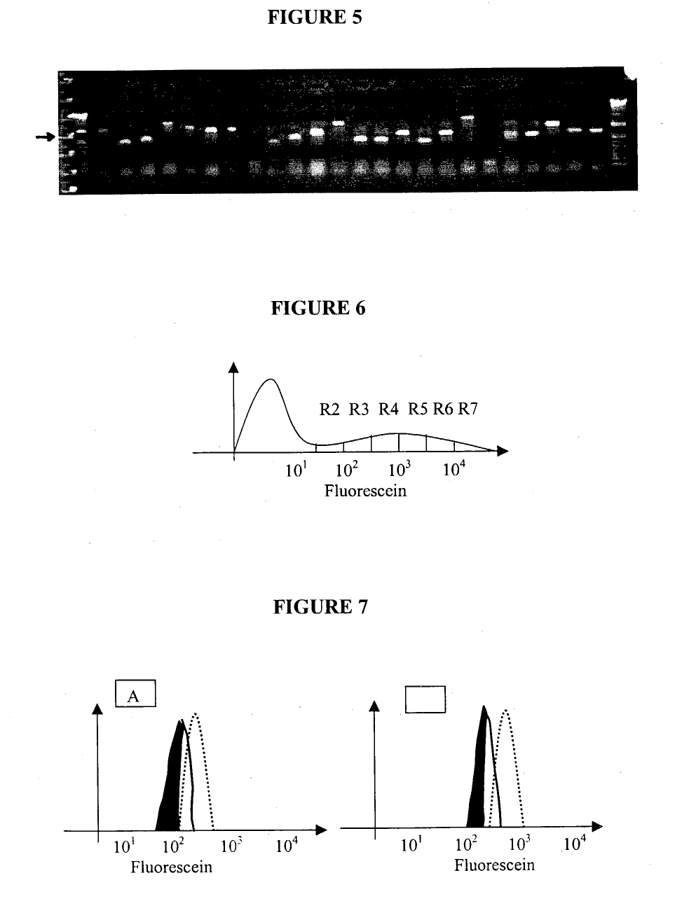

In order to verify library quality, we determined the titer of the library by calculating the transformants. The titer of the library was high: 106 transformants/ug of cDNA. In addition, we confirmed by PCR amplification that 95 percent of clones contained a cDNA insert larger than 800 bp. The libraries were thus deemed to be useful for screening short-lived proteins in mammalian cells. FIG. 5 shows an agarose gel verification of the sizes of cDNA inserts amplified from colonies randomly picked from the library of transformants.

2. Screening for Mammalian Cells Expressing GFP Fusion Proteins with Constitutive Short Half-Lives

Because GFP is an autofluorescent protein, its emission does not require cofactors or substrates. Therefore, GFP can be detected in real time in living cells without disrupting the cells. Furthermore, FACS can be used to fractionate cell populations according to the fluorescence intensity of individual cells.

We used 293T cells for expressing GFP-fusion libraries. 293T cells offer two potential advantages. First, the cells express the SV40 large T antigen, which results in high-copy extra-chromosomal replication of the vector so that plasmid can be recovered easily. Second, the host cells are recognized with high transfection efficiency. After we introduced the GFP-fusion libraries into the mammalian cells, the transfected cells were easily separated by FACS from nontransfected cells or cells transformed by nonproductive constructs.

We imposed selection for the desired cells according to the following two criteria: (1) cells that became dimmer after exposure to cycloheximide (CHX), a protein synthesis inhibitor and (2) become dimmer after a short treatment time, 2 hours. FIG. 8 is a scheme of the procedure used for isolating and characterizing short-lived proteins in this example.

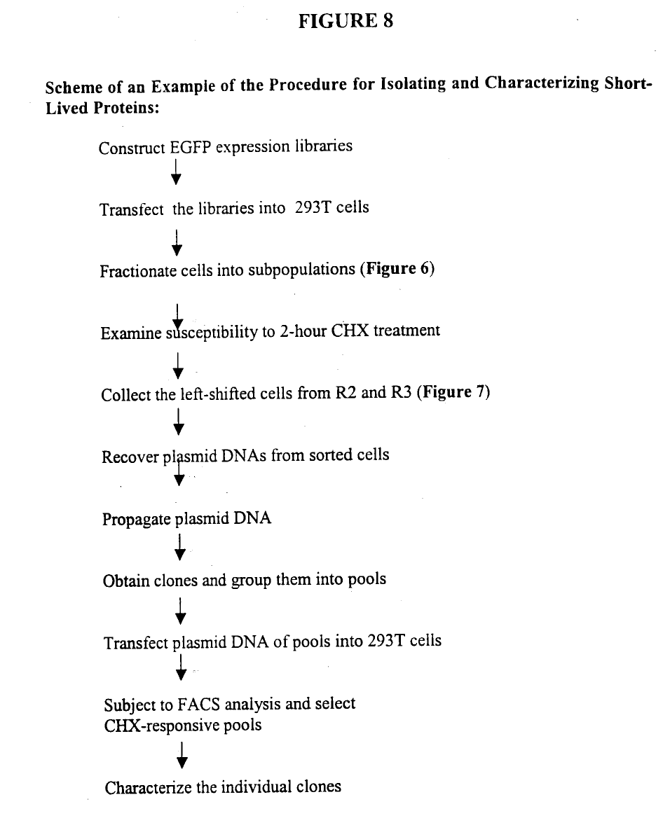

We began with a cell population that has an approximately log-normal fluorescence histogram distribution, with a working range of 1.5 to 3.5 logs. We used FACS fractionation to slice this population into six subpopulations (R2, R3, R4, R5, R6, R7) of ascending brightness, gating each on successive one-half log intervals of fluorescence (FIG. 6). After each subpopulation was divided into two, one subpopulation was treated with 100 ug/ml cycloheximide (CHX) for 2 hours and the other remained untreated. Subpopulations were then re-analyzed to determine whether they had retained a relative distribution consistent with the gating criteria used to obtain this narrow subpopulation and were susceptible to CHX treatment. We found that subpopulations of R3 and R4 ranging from log 2 to log 3 were susceptible to CHX treatment (FIG. 7, A: R3 population; and B: R4 population), while R5 and R6 ranging from log 4 to log 5, as well as R7, had no observable response to CHX treatment. The lack of susceptibility of the latter three subpopulations was most likely due to them expressing stable proteins and building up high fluorescence intensity.

We selected R4 for further screening. We collected 5×10⁵ cells from the shifted population. Plasmid DNAs were recovered from the sorted cells using Qiagen's mini-plasmid preparation kit with modifications. The plasmid DNAs were propagated by transforming into electrocompetent DH 10B cells. We obtained a total of 400 clones and possibly could obtain an additional 400 clones from R3 fraction. All of the individual clones were stored in 30 percent glycerol LB medium in a 96-well format. In order to perform second-round selection, we grouped 400 clones into 12 pools, each of which was composed of approximately 33 clones. The individual groups of clones were cultured and used for plasmid preparation. We transfected these 12 groups of plasmid DNA into 293T cells and subjected them to FACS analysis. The EGFP-C1 vector was used as a control. Because EGFP is a stable protein, its fluorescence intensity would not be changed by treatment with CHX. We found that eight of the 12 groups showed the decrease of the fluorescence intensity by 30 to 50 percent after two hours of CHX treatment. In four out of 12 groups, the change in fluorescence intensity was undetectable. There is one possibility for the lack of change in fluorescence intensity: the percentage of clones expressing short-lived proteins in these groups may be relatively small, so that the change in fluorescence intensity is barely detected.

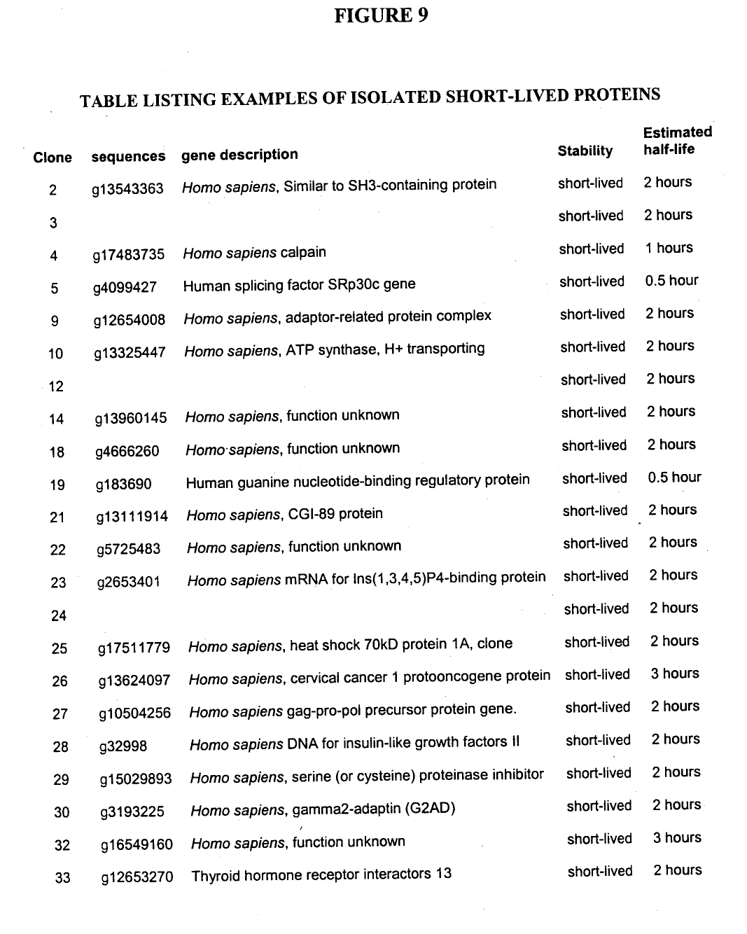

To pinpoint the individual clones with the desired property, we randomly chose a CHX-responsive group and characterized individual clones. We analyzed a total of 30 clones from the group by individually transfecting them and determining the half-life by FACS-based analysis of cycloheximide chase kinetics. Based on the calculation of 50% decrease in fluorescence of the clones, we estimated the half-life of each clone. We found out that 22 clones showed a decrease in fluorescence intensity ranging from 30 to 90 percent under the treatment of CHX for 2 hours, which was summarized in a table shown in FIG. 9. The 22 clones were sequenced and blasted against The National Center for Biotechnology Information (NCBI) public database. 19 of 22 were identifiable by BLAST search.

To the best of our knowledge, there are no published or publicly available sources that provide prior information on whether the proteins we have identified in fact turn over rapidly or not.

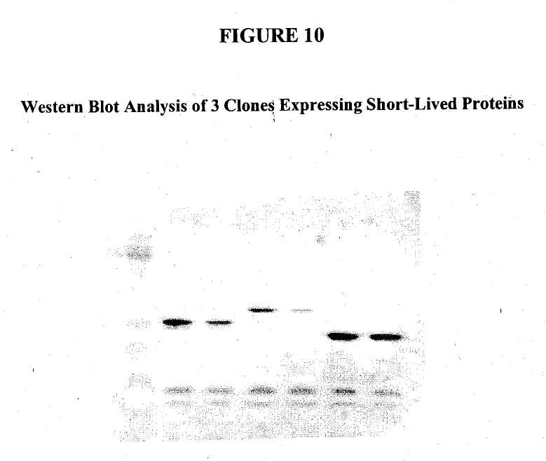

To directly check the stability of the candidate proteins, we did Western blot analysis of three clones that we randomly picked. 293T cells were transfected by the clones respectively and treated with or without CHX for 2 hours. The cell lystes were prepared from the cells and the proteins were separated by SDS gel electrophoresis. After transferring to membrane, the short-lived GFP fusion proteins were detected by polyclonal antibodies against GFP tag. As shown in the FIG. 10, all of these proteins degrade in the presence of CHX. However, EGFP protein itself was stable in the same condition (data not shown). The half-life of the proteins determined by Western blot analysis is similar to the fluorescent decay determined by FACS analysis, which indicates the concurrence between these two analyses. The western blot analysis confirmed the rapid turnover of these proteins that we identified with the FACS-based screening technology.

3. Construction of Protein Array of SH3-Domains

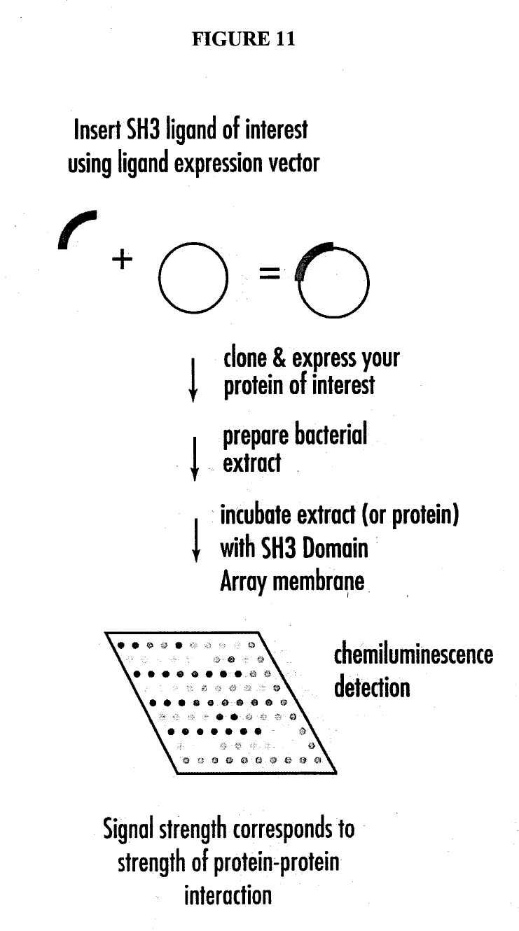

In this example, a membrane-based array of human SH3 sub-domains were constructed and screened for ligand-SH3 domain-specific interactions. Each SH3 domain binds to a conserved proline-rich motif on its ligand to initiate a protein interaction network. We have cloned 100 SH3 domains available from Genbank and have expressed the proteins in a GST-fusion format. Of these 100 proteins, we selected 38 fusions for constructing the protein array. To make the arrays, the coding sequences are PCR-amplified, cloned into a GST-based bacteria expression vector, and verified by sequencing. The recombinant GST-SH3 proteins were then expressed and purified. Finally, the purified proteins were spotted onto membranes to make the protein array. The principle of protein array analysis is illustrated in FIG. 11. This array-based technology has achieved proven results in high-throughput analysis of protein interactions.

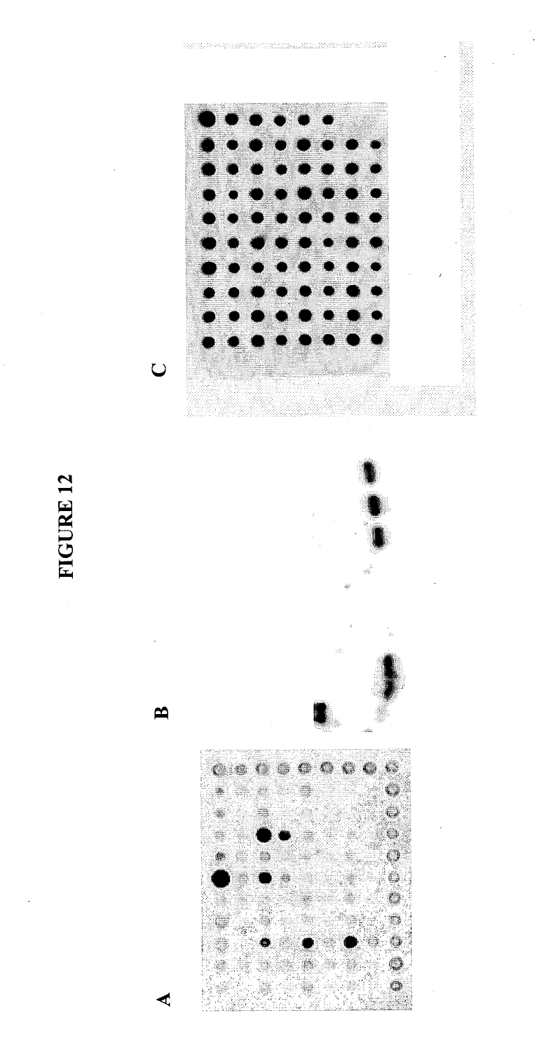

To demonstrate the array's utility, the well-studied binding site for SH3 domains from PI3 kinase was used as a ligand for array analysis (FIG. 12A). The cDNA sequence corresponding to PI3 kinase was cloned into an expression vector, and the cloned cDNA was expressed as a His-fusion protein that was incubated with the array membrane. Interactions between the ligand and the SH3 domains on the array were then detected with an antibody against the His tag.

As shown in FIG. 12A, the interaction of c-Src and related proteins with PI3K was detected, which is consistent with results published in the literature. The positive interactions were verified using a pull-down assay (FIG. 12B). As a control, the SH3 domain Array was incubated with anti-GST antibody and all spotted GST-fusion proteins were shown to be present in approximately equal amounts.

This technique described in this example can be used to construct arrays of short-lived proteins and antibodies against short-lived proteins of the present invention.

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

What is claimed is:

1. An oligonucleotide array, comprising:
a substrate; and
a plurality of oligonucleotide probes immobilized on a surface of the substrate such that different oligonucleotide probes are positioned in different defined regions on the surface, each of the different oligonucleotide probes comprising a binding region complimentary to a portion of a different gene encoding a short-lived protein,
wherein the short-lived protein has a half-life shorter than 24 hours in its native cellular environment.

2. The oligonucleotide array according to claim 1, wherein the short-lived protein has a half-life shorter than 12 hours in its native cellular environment.

3. The oligonucleotide array according to claim 1, wherein the short-lived protein has a half-life shorter than 4 hours in its native cellular environment.

4. The oligonucleotide array according to claim 1, wherein the short-lived protein has a half-life shorter than 2 hours in its native cellular environment.

5. The oligonucleotide array according to claim 1, wherein the oligonucleotide probes are a DNA, RNA, or PNA probes.

6. The oligonucleotide array according to claim 1, wherein each of the oligonucleotide probes comprises the DNA sequence of a portion of the cDNA of the short-lived protein.

7. The oligonucleotide array according to claim 1, wherein each of the oligonucleotide probes comprises the DNA sequence of a portion of the sense strand of the gene encoding the short-lived protein.

8. The oligonucleotide array according to claim 1, wherein each of the oligonucleotide probes comprises the DNA sequence of a portion of the 3′end of the sense strand of the gene encoding the short-lived protein.

9. The oligonucleotide array according to claim 1, wherein the length of each of the oligonucleotide probes is between 20-100 nt.

10. The oligonucleotide array according to claim 1, wherein the length of each of the oligonucleotide probes is between 40-80 nt.

11. The oligonucleotide array according to claim 1, wherein the length of each of the oligonucleotide probes is between 55-75 nt.

12. The oligonucleotide array according to claim 1, wherein each of the oligonucleotide probes is labeled with a detectable marker.

13. The oligonucleotide array according to claim 1, wherein the detectable marker is selected from the group consisting of biotin, radio-isotopes and fluorescent labels.

14. The oligonucleotide array according to claim 1, wherein the density of the array is lower than 1000.

15. The oligonucleotide array according to claim 1, wherein the density of the array is lower than 500.

16. The oligonucleotide array according to claim 1, wherein the density of the array is between 100-300.

17. The oligonucleotide array according to claim 1, wherein the density of the array is higher than 5,000.

18. The oligonucleotide array according to claim 1, wherein the density of the array is 1000-100,000.

19. The oligonucleotide array according to claim 1, wherein the diversity of the plurality of the oligonucleotide probes is higher than 50.

20. The oligonucleotide array according to claim 1, wherein the diversity of the plurality of the oligonucleotide probes is higher than 100.

21. The oligonucleotide array according to claim 1, wherein the diversity of the plurality of the oligonucleotide probes is between 100-2,000.

22. The oligonucleotide array according to claim 1, wherein the array is used to determine expression levels of the short-lived proteins.

23. The oligonucleotide array according to claim 1, wherein the array is-used to compare expression levels of the short-lived proteins in cells under normal and diseased condition.

24. A protein array, comprising:
a substrate; and
a plurality of short-lived proteins immobilized on a surface of the substrate such that different short-lived proteins are positioned in different defined regions on the surface, each of the different short-lived proteins having a half-time shorter than 24 hr in its native cellular environment.

25. The protein array according to claim 24, wherein the short-lived protein has a half-life shorter than 12 hours in its native cellular environment.

26. The protein array according to claim 24, wherein the short-lived protein has a half-life shorter than 4 hours in its native cellular environment.

27. The protein array according to claim 24, wherein the short-lived protein has a half-life shorter than 2 hours in its native cellular environment.

28. The protein array according to claim 24, wherein a portion of or the full-length protein of the short-lived protein is spotted on the array covalently or non-covalently.

29. The protein array according to claim 24, wherein the short-lived protein is fused with a non-short-lived protein.

30. The protein array according to claim 24, wherein the short-lived protein is a glutathione-s-transferase (GST) fusion protein.

31. The protein array according to claim 24, wherein the array is used to screen for agents that bind to the short-lived proteins on the array.

32 The protein array according to claim 31, wherein the agents are cellular proteins contained in cell lysates.

33. The protein array according to claim 32, wherein the cellular proteins contained in the cell lysates are labeled with a detectable marker.

34. An antibody array, comprising:
a substrate; and
a plurality of antibodies against short-lived proteins immobilized on a surface of the substrate such that different antibodies are positioned in different defined regions on the surface, each of the different short-lived proteins having a half-time shorter than 24 hr in its native cellular environment.

35. The antibody array according to claim 34, wherein the antibodies are polyclonal or monoclonal, human, non-human, chimeric, or humanized antibodies.

36. The antibody array according to claim 34, wherein the antibodies are fully assembled antibodies, Fab fragments, or single-chain antibodies.

37. The antibody array according to claim 34, wherein the short-lived protein has a half-life shorter than 12 hours in its native cellular environment.

38. The antibody array according to claim 34, wherein the short-lived protein has a half-life shorter than 4 hours in its native cellular environment.

39. The antibody array according to claim 34, wherein the short-lived protein has a half-life shorter than 2 hours in its native cellular environment.

40. The antibody array according to claim 34, wherein the array is used to screen for short-lived proteins that bind to the antibodies on the array.

41. The antibody array according to claim 40, wherein the short-lived proteins are cellular proteins contained in cell lysates.

42. The antibody array according to claim 41, wherein the cellular proteins contained in the cell lysates are labeled with a detectable marker.
